SEQUENCE LISTING (18 sequences)

SEQ ID NO: 1 - DNA, 20 bp, synthetic construct
  source 1..20: mol_type = other DNA
tcactaccggaaacagcgat

SEQ ID NO: 2 - DNA, 22 bp, synthetic construct
  source 1..22: mol_type = other DNA
ttcttaggtggttacattggct

SEQ ID NO: 3 - DNA, 20 bp, synthetic construct
  source 1..20: mol_type = other DNA
actttgcgtcgtgtgcttta

SEQ ID NO: 4 - DNA, 21 bp, synthetic construct
  source 1..21: mol_type = other DNA
gcactcgcgttaaactatcac

SEQ ID NO: 5 - DNA, 20 bp, synthetic construct
  source 1..20: mol_type = other DNA
cgctaacgccactttctacg

SEQ ID NO: 6 - DNA, 21 bp, synthetic construct
  source 1..21: mol_type = other DNA
gcagcacaagatcaagattgg

SEQ ID NO: 7 - DNA, 19 bp, synthetic construct
  source 1..19: mol_type = other DNA
ccttcaggtgttggaaagg

SEQ ID NO: 8 - DNA, 20 bp, synthetic construct
  source 1..20: mol_type = other DNA
caccttcacgcatatgacgc

SEQ ID NO: 9 - DNA, 20 bp, synthetic construct
  source 1..20: mol_type = other DNA
acataaactcgtcgggtggt

SEQ ID NO: 10 - DNA, 20 bp, synthetic construct
  source 1..20: mol_type = other DNA
agcgatcacacttgcgattg

SEQ ID NO: 11 - DNA, 20 bp, synthetic construct
  source 1..20: mol_type = other DNA
tcttgatggtatgcttccgc

SEQ ID NO: 12 - DNA, 20 bp, synthetic construct
  source 1..20: mol_type = other DNA
tcctttggatgatggtggtc

SEQ ID NO: 13 - DNA, 20 bp, synthetic construct
  source 1..20: mol_type = other DNA
tgcttctgtgatacggttgt

SEQ ID NO: 14 - DNA, 22 bp, synthetic construct
  source 1..22: mol_type = other DNA
tgaacacgatgagagaactgtt

SEQ ID NO: 15 - DNA, 20 bp, synthetic construct
  source 1..20: mol_type = other DNA
caacaagtcagccgtctagc

SEQ ID NO: 16 - DNA, 20 bp, synthetic construct
  source 1..20: mol_type = other DNA
cttgttccgccgctaattga

SEQ ID NO: 17 - DNA, 21 bp, synthetic construct
  source 1..21: mol_type = other DNA
tgtttcttagttgctgcaggt

SEQ ID NO: 18 - DNA, 20 bp, synthetic construct
  source 1..20: mol_type = other DNA
ttaggagacatgcacacggt

(19) United States
(12) Patent Application Publication
(10) Pub. No.: US 2026/0097207 A1
(43) Pub. Date: Apr. 9, 2026

(54) Bioelectronic Localized Antimicrobial Stimulation Therapy (BLAST) through selective excitability

(71) Applicants: The University of Chicago, Chicago, IL (US); THE REGENTS OF THE UNIVERSITY OF CALIFORNIA, Oakland, CA (US)

(72) Inventors: Bozhi Tian, Chicago, IL (US); Saehyun Kim, Chicago, IL (US); Gürol M. Süel, Chicago, IL (US)

(21) Appl. No.: 19/349,737

(22) Filed: Oct. 3, 2025

Related U.S. Application Data
(60) Provisional application No. 63/703,032, filed on Oct. 3, 2024.

(51) Int. Cl. A61N 1/36; A61N 1/04

(52) U.S. Cl. CPC A61N 1/36014; A61N 1/0408; A61N 1/0496

(57) ABSTRACT

The present disclosure relates to a method of electrically stimulating a resident bacteria on an epidermis of an animal for bioelectronic localized antimicrobial stimulation therapy (BLAST) through selective excitability and a wearable electroceutical device to aid in such methods.

### CROSS-REFERENCE TO RELATED APPLICATIONS

This application claims priority to U.S. Provisional Application No. 63/703,032, filed Oct. 3, 2024, the disclosure of which is hereby incorporated by reference in its entirety.

### STATEMENT REGARDING FEDERALLY SPONSORED RESEARCH

This invention was made with government support under W911NF-24-1-0053, and W911NF-22-1-0157 awarded by the Army Research Laboratory-Army Research Office, and 2121044 awarded by the National Science Foundation. The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Oct. 3, 2025, is named “24-1548-US Sequence Listing.xml” and is 20,480 bytes in size.

### FIELD OF THE DISCLOSURE

The disclosure is directed to novel methods and devices for electrical stimulation of microbes, like bacteria, to provide an antimicrobial effect. The methods and devices can be employed on different surfaces, such as the epidermis.

### BACKGROUND

Conventional electrical treatment for bacteria has primarily focused on electrostatic surface detachment of cells, high-voltage electroporation, and electrochemical biocide generation that irreversibly kills bacteria and damages the tissue around. Therefore, there remains a need for new devices and methodologies that harness the potential of electrical manipulation to provide new antimicrobial therapies.

### SUMMARY OF THE DISCLOSURE

As shown and described herein, the present disclosure relates to methods of electrically stimulating a resident bacteria on an epidermis of an animal for bioelectronic localized antimicrobial stimulation therapy (BLAST) through selective excitability and wearable electroceutical devices to aid in such methods.

In an embodiment of the present disclosure, a method of electrically stimulating a resident bacteria on an epidermis of an animal includes applying an acidic hydrogel to a region of the epidermis of the animal containing the resident bacteria, covering the region with a bioelectronic device, and administering an electrical stimulus through the bioelectronic device to the region. The electrical stimulus exerts an antimicrobial effect on the resident bacteria.

In various such embodiments, the acidic hydrogel includes a natural and/or synthetic polymer hydrogel with a pH of about 5.

In various such embodiments, the acidic hydrogel comprises pH 5-adjusted tragacanth gum.

In various such embodiments, the bioelectronic device includes a wearable electroceutical device having a first layer, the first layer being a wearable film dressing, and a second layer applied to an interfacing side of the wearable film dressing. The second layer includes a flexible polymer substrate and an electrode array integrated onto the flexible polymer substrate. The second layer is configured to be applied to the region via the first layer.

In various such embodiments, administering the electrical stimulus through the bioelectronic device to the region includes delivering alternating current to the bioelectronic device via a potentiostat connected to the bioelectronic device.

In various such embodiments, a voltage of 1.5 V_{ac} is delivered by the potentiostat to elicit a hyperpolarization response in the resident bacteria.

In various such embodiments, the antimicrobial effect includes a reduction in pathogenicity of the resident bacteria.

In various such embodiments, the reduction in pathogenicity of the resident bacteria includes a reduction in growth of the resident bacteria.

In various such embodiments, the reduction in growth of the resident bacteria includes at least one of: suppresses biofilm formation of the resident bacteria, reduces expression of virulence genes responsible for surface adhesion (SdrG), polysaccharide intracellular adhesin (icaB, icaC), protease (Clp), antibiotic resistance (MrSA), and virulence regulation (SarA), and reduces colonization capability of the resident bacteria.

In various such embodiments, the resident bacteria includes at least one of: Staphylococcus epidermidis, Staphylococcus capitis, and Staphylococcus saprophyticus.

In various such embodiments, the animal is a mammal.

In an embodiment of the present disclosure, a wearable electroceutical device includes a first layer. The first layer is a wearable film dressing. The wearable electroceutical device includes a second layer applied to an interfacing side of the wearable film dressing. The second layer includes a flexible polymer substrate and an electrode array integrated onto the flexible polymer substrate. The second layer is configured to be applied to a mammalian epidermis via the first layer. An acidic hydrogel interlayer is configured to be disposed between the second layer and the mammalian epidermis.

In various such embodiments, the electrode array includes gold electrodes.

In various such embodiments, the flexible polymer substrate includes a flexible polyimide substrate.

In various such embodiments, the electrode array is interdigitated. The electrode array enables electric potential localization between adjacent fingers of the electrodes in the electrode array.

In various such embodiments, the acidic hydrogel interlayer includes a natural and/or synthetic polymer hydrogel with a pH of about 5.

In various such embodiments, the acidic hydrogel interlayer sensitizes resident bacteria on the mammalian epidermis to electrical excitation.

In various such embodiments, the resident bacteria includes at least one of: Staphylococcus epidermidis, Staphylococcus capitis, and Staphylococcus saprophyticus.

In various such embodiments, a potentiostat connected to the wearable electroceutical device is configured to deliver alternating current to the wearable electroceutical device.

In various such embodiments, the potentiostat is configured to deliver a voltage of about 1.5 V_{ac} to the wearable electroceutical device to elicit a hyperpolarization response in the resident bacteria.

### BRIEF DESCRIPTION OF THE DRAWINGS

In the figures, the following symbols were used to indicate significance levels: “n.s” for non-significant (P>0.05), “*” for P<0.05, “**” for P<0.01, “***” for P<0.001, and “****” for P<0.0001.

FIGS. 1A-1E depict an interdigitated stimulation platform that enables the assessment of bacterial excitability in response to electrical stimulation. FIG. 1A depicts opportunistic infections by S. epidermidis that serve as a leading source of healthcare-associated infections. It is investigated if an excitatory response in S. epidermidis through electrical stimulation can be induced, enabling bioelectrical modulation. FIG. 1B depicts schematics of the electrical simulation device used to study the excitability of S. epidermidis. FIG. 1C depicts a photograph of the electrical stimulation device. Scale bar, 5 mm. FIG. 1D depicts a zoomed-in image of interdigitated gold microelectrodes. FIG. 1E depicts phase contrast (left) and fluorescence image of Thioflavin T (ThT) stained S. epidermidis (right) located between the interdigitated gold electrode array. ThT reports on the membrane potential. The color bar illustrates the intensity range of ThT-stained cells. Scale bar, 20 μm.

FIGS. 2A-2H demonstrate how epidermal pH confers electrical excitability to S. epidermidis. FIG. 2A depicts ThT intensity traces that show that electrical stimulation hyperpolarizes S. epidermidis only at external pH (pHₑ)=5. Fluorescence intensity is calculated as log (F/FO), where F is the fluorescence and FO is the fluorescence at the resting state (see also FIG. 8 for single cell traces.) FIG. 2B depicts confocal images that show that electrical stimulation elicits hyperpolarization at pHₑ=5 but not at pHₑ=7.4. The pH of 7.4 represents the unmodified pH of tryptic soy broth medium where the bacteria grow most optimally. Scale bar, 5 μm. The color bar illustrates the intensity range of ThT-stained cells. FIG. 2C depicts an intensity histogram of ThT stained cells at rest and post-stimulation at pHₑ=5 (n≥300). FIG. 2D depicts pHrodo intensity traces that show that electrical stimulation acidifies the cytoplasm of S. epidermidis only at pHₑ=5. pHrodo reports on the internal pH, with its fluorescence increasing at a lower pH. (See also FIG. 8 for single cell traces.) FIG. 2E depicts confocal images show that electrical stimulation induces acidification of cytoplasm at pHₑ=5, but not at pHₑ=7.4. Scale bar, 5 μm. The color bar illustrates the intensity range of pHrodo-stained cells. FIG. 2F depicts an intensity histogram of pHrodo stained cells at rest and post-stimulation at pHₑ=5 (n≥300). FIG. 2G depicts the maximal transmembrane proton gradient, ΔH⁺, exists near pHₑ=5, which could be abolished by the addition of 150 μM carbonyl cyanide m-chlorophenyl hydrazone (CCCP, Cayman Chemical) (n=4). Experimentally, cells are electrically excitable only near the epidermal pH where substantial ΔH⁺ is present. FIG. 2H depicts that, upon addition of CCCP (150 μM) and Nigericin (3 μM), S. epidermidis shows greater cytoplasmic acidification at pHₑ=5 compared to pHₑ=7.4. BCECF-AM reports on internal pH, with its fluorescence decreasing at a lower pH. The arrow denotes the point of addition (n=4). All data are presented as mean values±SD.

FIGS. 3A-3F demonstrate that epidermal pH confers selective excitability to two other skin-residing opportunistic pathogens. FIG. 3A depicts mean ThT intensity traces that show that electrical stimulation hyperpolarizes S. capitis at pHₑ=5, but not at pHₑ=7.4 (n=5). FIG. 3B depicts confocal images that show that electrical stimulation at pHₑ=5 hyperpolarizes S. capitis, but not at pHₑ=7.4. Scale bar, 20 μm. The color bar illustrates the intensity range of ThT-stained cells. FIG. 3C depicts that the transmembrane proton gradient of S. capitis at pHₑ=5 is significantly larger than at pHₑ=7.4 (n=4). FIG. 3D depicts ThT intensity traces that show that electrical stimulation hyperpolarizes S. saprophyticus at pHₑ=5, but not at pHₑ=7.4 (n=5). FIG. 3E depicts confocal images that show that electrical stimulation at pHₑ=5 hyperpolarizes S. saprophyticus, but not at pHₑ=7.4. Scale bar, 20 μm. The color bar illustrates the intensity range of ThT-stained cells. FIG. 3F depicts that the transmembrane proton gradient of S. saprophyticus at pHₑ=5 is significantly larger than at pHₑ=7.4 (n=4). All data are presented as mean values±SD.

FIGS. 4A-4B demonstrate population response of S. epidermidis that reveals the suppressive effect of electrical stimulation. FIG. 4A depicts that electrical stimulation reduces the growth and ATP levels in S. epidermidis at pHₑ=5, but not at pHₑ=7.4 (n=4). FIG. 4B depicts that electrical stimulation at pHₑ=5 reduces virulence gene expression in S. epidermidis, measured by the quantitative reverse transcription polymerase chain reaction (n=4). The fold change was normalized relative to the expression level of guanylate kinase (gmk) housekeeping gene. All data are presented as mean values±SD.

FIGS. 5A-5F demonstrate that elective excitability enables programmable biofilm suppression. FIG. 5A depicts a mean intensity trace of S. epidermidis co-stained with ThT and Gentamicin Texas red (GTTR). Electrical stimulation at pHₑ=5 results in hyperpolarization accompanied by the accumulation of gentamicin (n=5). Scale bar, 5 μm. The color bar illustrates the intensity range of GTTR-stained cells. FIG. 5B depicts that electrical pretreatment at pHₑ=5 enhances the suppressive effect of sublethal gentamicin (30 ug/mL) towards S. epidermidis biofilm formation (n≥4). Stimulation was abbreviated as stim. FIG. 5C depicts phase contrast images that show electrical pretreatment at pHₑ=5 combined with a sublethal gentamicin ceases bacterial growth. Scale bar, 20 μm. FIG. 5D depicts phase contrast images of S. epidermidis biofilm after 18 h of periodic electrical stimulation, applied every 10 minutes. Periodic stimulation abolishes biofilm formation at pHₑ=5 but not at pHₑ=7.4. Scale bar, 20 μm. FIG. 5E depicts that periodic electrical stimulation abolishes biofilm growth at pHₑ=5 (n=4). FIG. 5F depicts that periodic electrical stimulation does not reduce biofilm growth at pHₑ=7.4 (n=4), indicating a non-lethal bioelectrical stimulation condition. All data are presented as mean values±SD.

FIGS. 6A-6G demonstrate that Bioelectronic Localized Antimicrobial Stimulation Therapy (BLAST) reduces colonization of porcine skin by S. epidermidis. FIG. 6A depicts a schematic diagram showing structural components of the electroceutical skin patch used to stimulate S. epidermidis. FIG. 6B depicts a photograph showing wearability and flexibility of the skin patch. Scale bar, 1 cm (top), 40 μm (bottom). FIG. 6C depicts a 3D reconstruction of confocal z-stack images shows hyperpolarization of ThT-stained S. epidermidis upon electrical stimulation. Scale bar, 20 μm. The color bar illustrates the intensity range of ThT-stained cells.

FIG. 6D depicts a ThT intensity histogram shows population-wide hyperpolarization response clicited upon electrical stimulation of S. epidermidis (n≥600). FIG. 6E depicts that BLAST significantly reduces the colony forming units of S. epidermidis on porcine skin after 18 h (n=5). Small panels show visible reduction in the opacity of S. epidermidis collected and cultured from porcine skin. Scale bar, 1 cm. FIG. 6F depicts that BLAST significantly reduces the S. epidermidis biofilm coverage on porcine skin after 18 h (n≥5). FIG. 6G depicts SEM imaging that shows that BLAST decreases S. epidermidis biofilm coverage on the porcine skin surface. Scale bar, 5 μm. All data are presented as mean values±SD.

FIGS. 7A-7B depict device designs for electrical stimulation of opportunistic pathogens. FIG. 7A depicts a device for microscopy-based evaluation of microbial electrophysiology. The device includes a 0.17 mm glass coverslip substrate, an acrylate plastic grid, and gold. FIG. 7B depicts a flexible device for electrical stimulation of porcine skin, having gold and a polyimide substrate. The numbers are in micrometers (μm). FIG. 7C depicts characterization of interdigitated microelectrodes. Left: image from LEXT OLS 5100 confocal microscope. Right: image from SEM. Scale bar, 10 μm.

FIG. 8 depicts a COMSOL Multiphysics® simulation of the electric potential distribution between interdigitated electrodes, immediately after applying relative voltage of 1.5V. Arrows indicate the electric field direction.

FIGS. 9A-9B demonstrate optimization of electrical stimulation conditions. FIG. 9A depicts a ThT intensity trace of electrically stimulated S. epidermidis at pH 5 (n=4). FIG. 9B depicts a fluorescence image of ThT stained S. epidermidis before and after stimulation. Scale bar, 20 μm. Excitatory response was not observed at 40 mVpp/μm. Increasing the field strength to 60 m Vpp/μm resulted in subtle membrane potential change. 75 mVpp/μm elicited the most measurable response. Field strength was not increased further to prevent potential damages to the electrode. While the frequency of 1 Hz elicited changes in membrane potential, the response was weaker than at 100 Hz. Altogether, 75 m Vpp/μm at 0.1 kHz AC was selected as the stimulation condition. This condition strongly aligns with the parameters used in Stratford, J. P. et al. pioneering work on B. subtilis stimulation.

FIG. 10 demonstrates electrical stimulation at pH 5 causes a hyperpolarization of S. epidermidis, shown by tetramethylrhodamine (TMRM) staining. S. epidermidis was stained with the TMRM membrane voltage indicator (200 nM) which shows increase in fluorescence after electrical stimulation, indicating hyperpolarization (n>1000). Scale bar, 20 μm.

FIGS. 11A-11B depict single cell intensity traces of ThT and pHrodo stained cells upon electrical stimulation at pH 5. Population-wide responses were observed (n≥14).

FIG. 12 demonstrates that S. epidermidis grows optimally at pH 7.4. The optical density of S. epidermidis was adjusted to OD₆₀₀-0.27 in TSB medium and incubated for 4 hours at 37° C. in a shaker incubator before taking the final OD₆₀₀ measurements. The results indicate that pH 5 is an unfavorable pH for bacterial growth. S. epidermidis grows most optimally at pH 7.4, which is the unmodified pH of the TSB medium used for routine bacterial culture (n=3).

FIG. 13 demonstrates that electrical stimulation does not permeabilize the bacterial membrane. Live cells are stained with SYTO9, while dead cells with a compromised membrane are stained with propidium iodide. No significant decrease in viability was observed 30 minutes after the electrical stimulation (n=3).

FIGS. 14A-14C demonstrate that ThT intensity trace of electrically stimulated S. epidermidis at pH 5 shows recovery of membrane potential and growth. FIG. 12A depicts an intensity trace of ThT-stained S. epidermidis. The electrical stimulation was applied at the 20 minutes timepoint, reflected by the initial increase in ThT fluorescence intensity (n=50). FIG. 14B depicts single-cell ThT intensity traces (n=50). FIG. 14C depicts that, upon electrical stimulation at 20 min, S. epidermidis recovers its membrane potential over time and resumes growth. 3 μM ThT was used for the long-term timelapse. Scale bar, 20 μm.

FIGS. 15A-15B depict an intensity trace of fluorescein dye that shows that no change in external pH occurs upon electrical stimulation. FIG. 15A depicts calibration of fluorescein intensity verses external pH (n=4). FIG. 15B depicts that no change in fluorescein intensity occurred upon electrical stimulation at pH 5, showing that external pH does not fluctuate during stimulation.

FIG. 16 demonstrates that an excitation laser for ThT and pHrodo alone does not influence their membrane potential and internal pH. No change in fluorescence intensity was observed for ThT and pHrodo stained cells without electrical stimulation.

FIG. 17 demonstrates a correlation between the transmembrane pH gradient (ΔpH) and membrane potential. As the ΔpH decreases, membrane potential hyperpolarizes, evidenced by increased ThT fluorescence. This compensation of ΔpH through alterations in membrane potential is well documented across various bacteria as a homeostatic mechanism to maintain the proton motive force (PMF), approximated as PMF (mV)=Δψ−(59×ΔpH), where Δψ is the membrane potential. During electrical excitation, pHrodo staining indicates decrease in internal pH (pHᵢ) while external pH (pHₑ) remains unchanged (See also FIG. 12), resulting in reduction of ΔpH. Therefore, a hyperpolarization response was observed, consistent with the experimental and literature trends. ΔpH was measured by BCECM-AM staining (n≥4).

FIGS. 18A-18B demonstrate electrical stimulation of S. epidermidis at intermediate PH levels. FIG. 18A depicts that a mean ThT intensity trace shows that S. epidermidis can be electrically excited at pH 5.5 to hyperpolarize the membrane potential (n=3). FIG. 18B depicts that a mean ThT intensity trace shows that S. epidermidis cannot be electrically excited at pH 4.5 to hyperpolarize the membrane potential (n=3). Scale bar, 20 μm.

FIG. 19 depicts a mean ThT intensity trace upon electrical stimulation at pH=5. The addition of 150 μM CCCP mutes the hyperpolarization response (n=5).

FIGS. 20A-20B demonstrate an electrical response of E. coli to stimulation. FIG. 20A depicts a mean ThT intensity trace of E. coli with or without electrical stimulation (n=3).

FIG. 20B depicts fluorescence images of ThT-stained E. coli before and after stimulation. While the excitation laser alone (without stimulation, line) does not affect ThT fluorescence, electrical stimulation elicits a rapid hyperpolarization response in E. coli. The results indicate that the stimulation may be used to control another gram-negative opportunistic pathogen. Scale bar, 20 μm.

FIG. 21 demonstrates that fluorescence intensity of Gentamicin Texas red increases upon addition of Nigericin. Nigericin is an ionophore that mimics the effect of electrical stimulation by acidifying intracellular pH and hyperpolarizing the membrane potential (n>170). Scale bar, 10 μm.

FIG. 22 demonstrates that periodic electrical stimulation at pH 5 results in localized inhibition of S. epidermidis biofilm formation. Biofilm inhibition only occurs at interdigitated electrode arrays. Outside the electrode region, biofilm growth occurs. Scale bar, 40 μm.

FIG. 23 demonstrates that significantly higher voltages are required for biofilm suppression in the absence of selective excitability. At pH 7.4, where the excitatory response was absent, a voltage as high as 9.5V_{ac} was required to achieve around 90% biofilm inhibition. Moreover, severe electrode degradation was observed at these high voltages. This demonstrates the advantages of our method, which leverages the selective excitability of S. epidermidis to achieve 99% biofilm suppression with a voltage as low as 1.5 V_{ac}, while maintaining device stability over repeated stimulation cycles (n=3). Scale bar, 40 μm.

FIGS. 24A-24B demonstrate that tragacanth gum can restore altered skin pH. FIG. 24A depicts hue, saturation, and brightness of the MQuant® pH test strip in contact with pH 7.4 PBS, PBS-soaked porcine skin, pH 5 tragacanth gum, and PBS-soaked porcine skin after treated with the tragacanth gum (n=5). FIG. 24B depicts a photograph of the MQuant® pH test strip upon contact with each sample. The results show that PBS-soaked porcine skin, which has an elevated pH level, acquires acidic pH of tragacanth gum upon treatment. This indicates that tragacanth gum can lower elevated pH levels in unhealthy skin to the acidic level, sensitizing the resident bacteria to electrical treatment.

FIG. 25 demonstrates stability of the bioelectronic skin patch. Electrochemical impedance spectroscopy shows the stability of device impedance over 500 cycles of periodic electrical stimulation, which could last 5 days. Each datapoint is an average of five repeated measurements.

FIG. 26 demonstrates efficacy of the bioelectronics skin patch on other medically relevant surfaces. When the identical device was applied to the silicone and ultra-high molecular weight polyethylene surfaces (UHMWPE) commonly used for catheter and prosthetic implants, periodic electrical stimulation significantly decreased colony forming units of S. epidermidis on the surfaces.

### DETAILED DESCRIPTION

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the meaning commonly understood by one of ordinary skill in the art. Unless otherwise required by context, singular terms shall include the plural and plural terms shall include the singular.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

In some embodiments, percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

### I. Overview

In general, the present disclosure relates to a method of electrically stimulating a resident bacteria on an epidermis of an animal for bioelectronic localized antimicrobial stimulation therapy (BLAST) through selective excitability and a wearable electroceutical device to aid in such methods. The wearable bioelectronic patch can apply electrical stimulation to excite bacteria on human skin. This excitatory response reduces the pathogenicity of the resident bacteria, making them less harmful.

Specifically, it was discovered that an acidic skin pH can render skin-residing bacteria electrically excitable. Leveraging this discovery, an acidic hydrogel layer was incorporated between the flexible bioelectronic patch and the skin. This hydrogel sensitizes resident bacteria to electrical excitation. By applying electrical stimulation, bacterial colonization on porcine skin and other medically relevant surfaces was successfully inhibited.

This technology offers an antibiotic-free approach to controlling Staphylococcus epidermidis, an opportunistic pathogen that is a leading cause of clinical infections. The localized and programmable electrical stimulation provides an advantage over traditional antibiotics, which can have systemic side effects and contribute to antibiotic resistance. Conventional electrical treatment for bacteria has primarily focused on electrostatic surface detachment of cells, high-voltage electroporation and electrochemical biocide generation that irreversibly kills bacteria and damages the tissue around. This contrasts with electroceuticals targeting mammalian tissues (i.e., neurons) that leverage innate cellular excitability to elicit non-lethal and trainable bioelectrical responses. The electrical treatment for bacteria is less developed compared to those acting on mammalian systems, as bacterial electrophysiology is still being elucidated.

This technology harnesses an innate electrophysiological response of bacteria (i.e., selective excitability) to programmably control bacterial pathogenicity, with a very low voltage that is safe for humans. Without harnessing the excitatory response, or by resorting to the old methods for electrical treatment, similar levels of bacterial inhibition can only be achieved with damaging and high voltage levels.

Harnessing the natural excitability in mammalian tissues has driven the development of drug-free therapies such as electroceuticals. However, it has been unclear if bacterial residents on the human body are equally excitable, and if their excitability can also be leveraged for drug-free bioelectronic treatment. Using a tailored microelectronic platform for microbial electrophysiology, the electrical excitability of Staphylococcus epidermidis, a skin-residing opportunistic pathogen that causes widespread clinical infections, was examined. It was discovered that a non-lethal electrical stimulus elicits an excitatory response in S. epidermidis, inducing reversible changes in membrane potential. Intriguingly, S. epidermidis and other skin pathogens were only excitable when subjected to epidermal pH. This selective excitatory response at the skin pH enabled programmable suppression of biofilm formation in S. epidermidis. Lastly, a wearable electroceutical patch was engineered that can sensitize resident bacteria to electrical excitation, suppressing colonization of S. epidermidis on a porcine skin model. This shows that the innate excitability of resident bacteria can be selectively activated and leveraged for drug-free bioelectronic control.

Epidermal pH confers electrical excitability to skin-residing opportunistic pathogens, enabling drug-free, bioelectronic control of bacterial virulence factors.

### II. Selective Excitability of Relevant Bacteria

Leveraging the innate electrical excitability of eukaryotic cells has enabled bioelectrical modes of tissue modulation, providing alternatives to drug-based therapies. Examples include electroceuticals, which are therapeutic bioelectronic devices that stimulate the body's naturally excitable circuits. Bioelectronic devices such as cardiac pacemakers, retinal prostheses, and nerve stimulators, for example, demonstrate how understanding and harnessing natural excitability in tissues can bring extensive health benefits. In these devices, well-known excitable cells such as neurons have been the main target of modulation. However, the recent discovery of innate electrical excitability in microbes suggests a potential for the bioelectrical control of commensal bacteria that are crucial to human health. To target bacteria inhabiting the human body, it would be preferable to use low voltage stimulation to modulate bacterial physiology and pathology similarly to how electroceuticals are used in human medicine.

There is a need to effectively control the bacteria residing on the human body, as many of them can become opportunistic pathogens under certain conditions. For example, Staphylococcus epidermidis, commonly found on healthy skin, typically promotes tissue homeostasis and aids in wound healing. However, factors such as compromised skin barrier, immunosuppression, and biofilm formation can shift its behaviour towards pathogenicity. In the virulent state, S. epidermidis is a significant contributor to neonatal morbidity and is the second leading cause of hospital infections due to the formation of antibiotic-resistant biofilm in clinical implants. Moreover, the severity of dermatological disorders like atopic dermatitis and scalp seborrheic dermatitis (i.e. dandruff) is linked to an over-proliferation of S. epidermidis on the skin. Effective management of opportunistic pathogens is a major challenge that, if overcome, could have far-reaching implications.

Bioelectronic control of opportunistic pathogens can offer unique advantages over antibiotic treatments. Antibiotics carry widespread side effects, such as nausea, drug-fever, and nephrotoxicity. Repeated exposure to antibiotics increases the risk factor for chronic inflammatory disorders and contributes to antimicrobial resistance, a global health threat.

Three strains of S. epidermidis that are pan-resistant to all classes of antibiotics have emerged, exposing the fragility of drug-based methods. Unlike drugs, bioelectrical treatments can allow for localized and targeted therapy, thereby minimizing systemic side-effects. Bioelectronic methods can also be applied automatically with programmable stimulation parameters that can be optimized for individual patients to enable personalized medication.

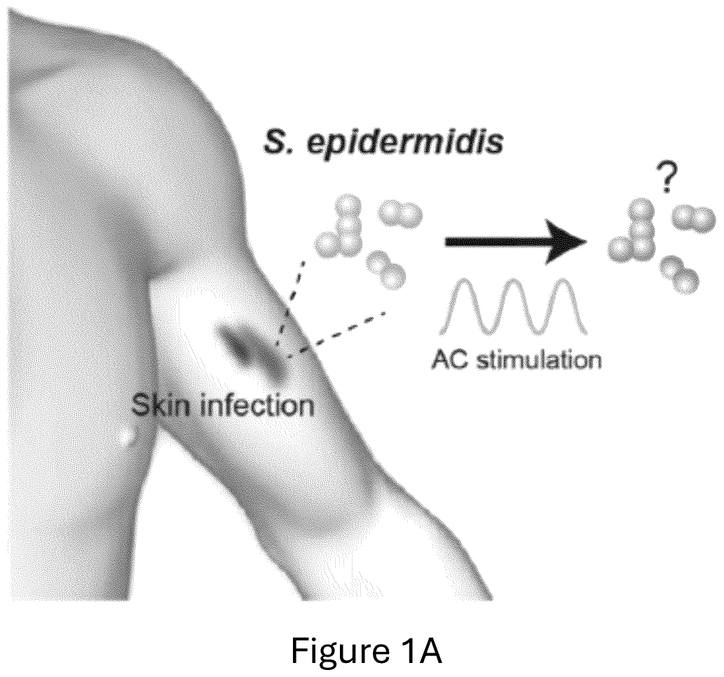

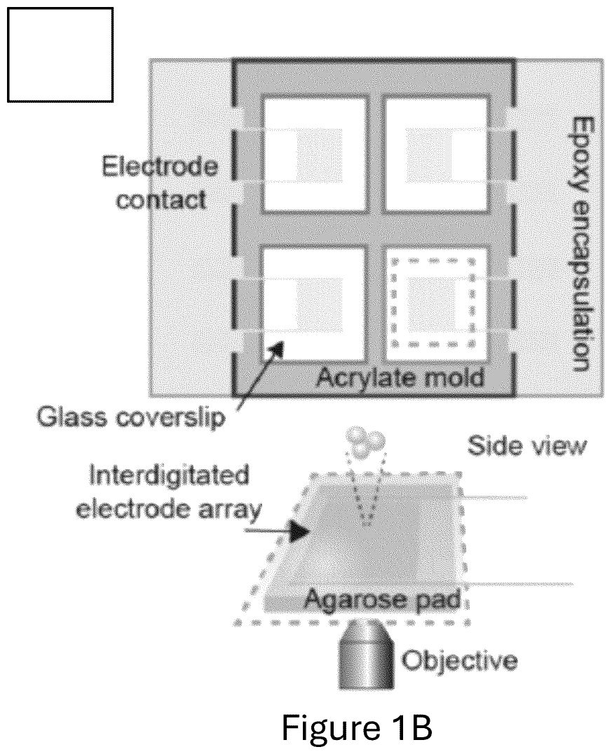

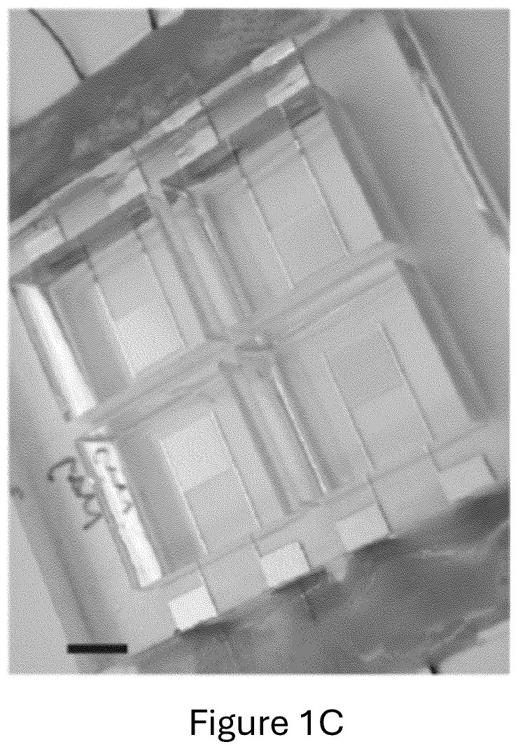

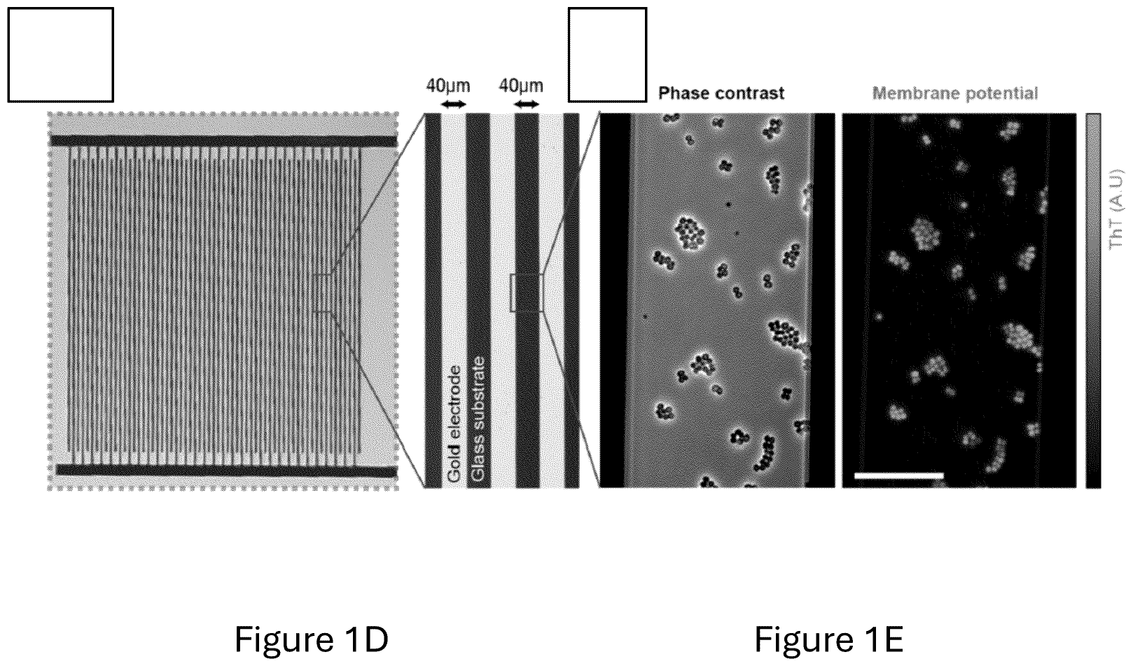

Despite being a potential drug-free alternative, existing electrical treatment for bacteria is less developed compared to those acting on mammalian systems, as bacterial electrophysiology is still being elucidated. Conventional electrical treatment for bacteria has primarily focused on electrostatic surface detachment of cells, high-voltage electroporation and electrochemical biocide generation that irreversibly kills bacteria. This contrasts with electroceuticals targeting mammalian tissues that leverage innate cellular excitability to elicit non-lethal and programmable bioelectrical responses. Given that bioelectrical potential is closely related to growth, virulence, and antibiotic resistance in bacteria, bioelectronic devices that exploit excitatory responses in opportunistic pathogens could offer a powerful handle to steer bacterial physiology in favorably, as is shown in FIG. 1A.

Several gaps in knowledge must be filled to engineer devices that can exploit excitatory responses in opportunistic pathogens. Among the human microbiota, only two species have been conclusively shown to be electrically excitable, demonstrating the ability to generate changes in membrane potential in response to electrical stimulation. Specifically, E. coli and B. subtilis, which reside in the gastrointestinal tract, exhibit changes in membrane potential when electrically stimulated. This number of reported electrically excitable species is remarkably small compared to the thousands of bacterial species that reside in human bodies.

Consequently, molecular mechanisms underlying bacterial electrophysiology have only been elucidated for a few model organisms such as B. subtilis, and far less is known about more medically relevant species. Furthermore, the long-term effects, as well as the clinical relevance of excitatory responses in opportunistic pathogens remain unclear. This gap in understanding has impeded the development of wearable or implantable bioelectronics designed to control resident bacteria by harnessing their innate electrical excitability.

A microelectronic platform was engineered to examine the electrical excitability of the skin-residing opportunistic pathogen, S. epidermidis. The findings showed that S. epidermidis can be excited by a non-lethal electrical stimulus, causing reversible changes in membrane potential. Intriguingly, S. epidermidis and other skin pathogens were excitable only when subjected to an acidic epidermal pH. Hence, the term “selective excitability” is used to describe excitatory behavior that only occurs under select conditions, such as in this case, the epidermal pH. The bioelectrical stimulation inhibited growth, virulence gene expression, and biofilm formation in S. epidermidis. Leveraging these findings, an electroceutical patch was developed that exploits selective excitability of S. epidermidis, preventing skin colonization using a voltage that is safe and imperceptible to humans. This discovery and utilization of selective excitability in bacteria enables drug-free bioelectronic control of opportunistic pathogens.

### III. Development of the Electrical Excitability Methods and Related Apparatuses

### A Tailored Device Platform Enables the Assessment of Bacterial Excitability in Response to Electrical Stimulation

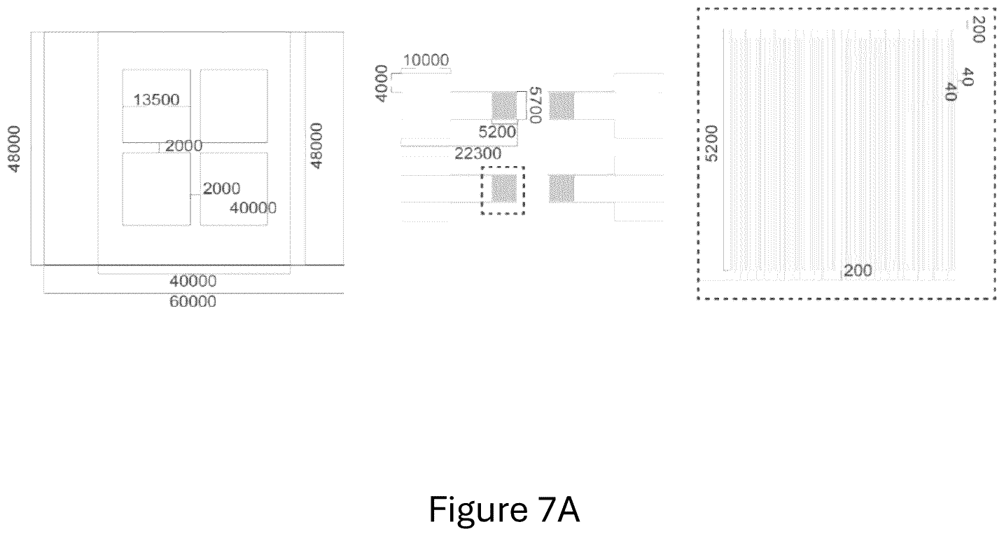

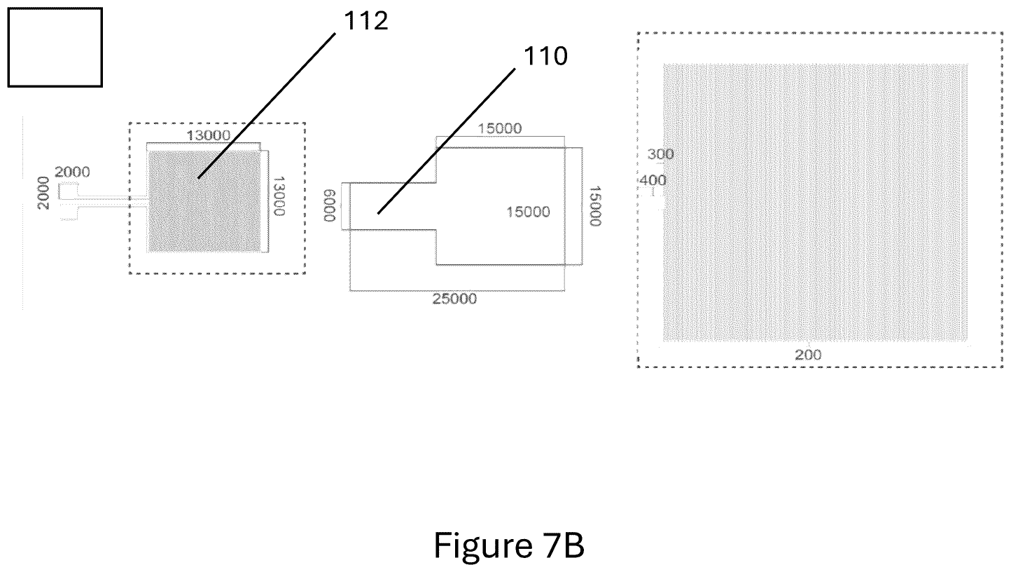

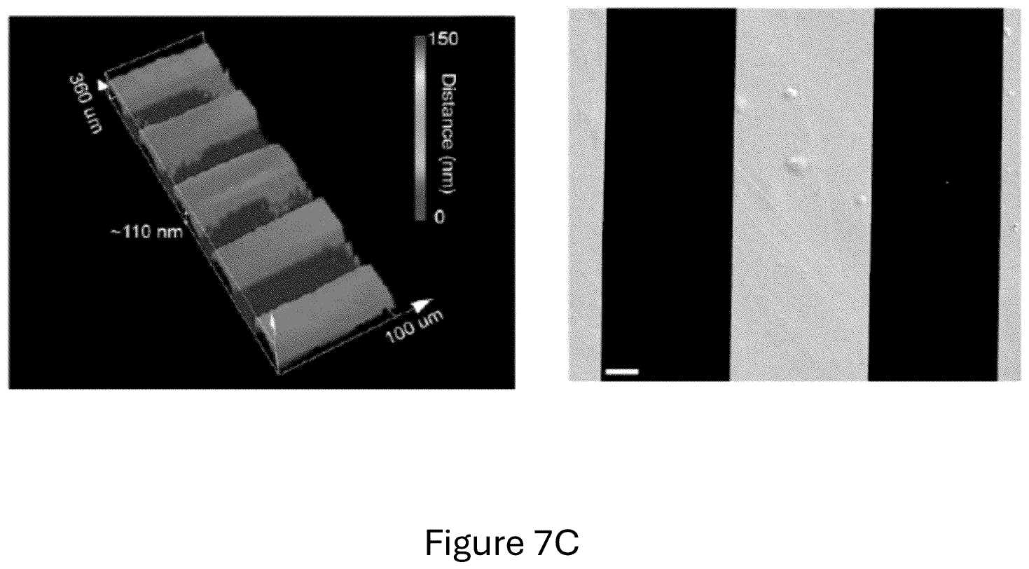

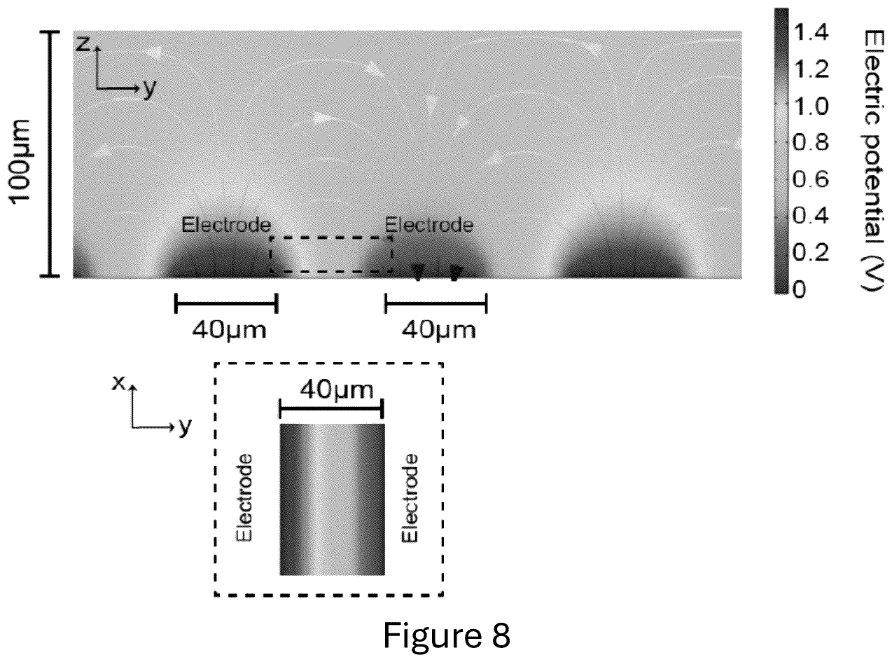

To observe the effects of electrical stimulation at single-cell resolution, a customized setup for bacterial electrophysiology was developed, as shown in FIGS. 7A-7C. Interdigitated gold electrodes were fabricated on a 0.17 mm glass coverslip, compatible with the use of 63× and 100× microscope objectives that have a short working distance, (FIGS. 1B, 1C). Interdigitated designs enable electric potential localization between adjacent fingers for effective cell modulation (FIG. 8), while gold provides superior biocompatibility. After inoculating the bacteria on agarose pads, these were placed on the electrode surface, and a potentiostat was used to deliver alternating current at specified frequencies and voltages (see below for details). The electrodes were designed with a width and gap of 40 μm (FIG. 1D), suitable for the small size of S. epidermidis, which ranges from approximately 1 μm to 2 μm. Incorporating a 2×2 grid within a single device enabled performance of parallel experiments with varying conditions, facilitating statistical analysis. The customized setup allowed for delivery of exogenous electrical stimuli and monitoring of electrophysiological changes in bacteria through fluorescence and phase-contrast microscopy (FIG. 1E).

Epidermal pH Confers Electrical Excitability to S. epidermidis

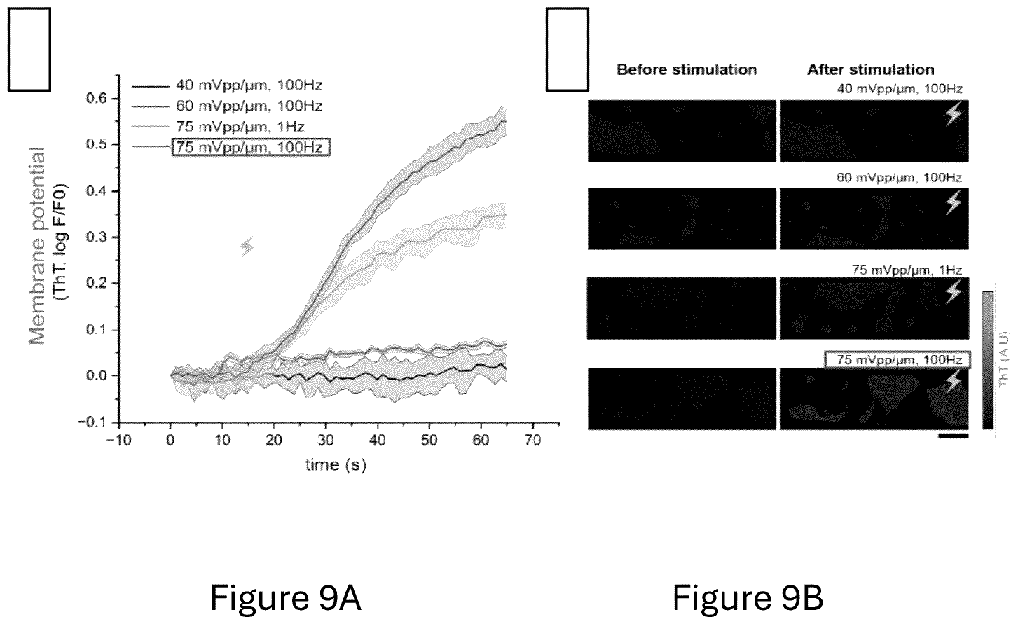

Skin-residing bacteria are exposed to diverse pH conditions, with healthy skin typically ranging from 4.7 to 5.2, and skin with atopic dermatitis from 5.5 to 5.9. Acne vulgaris presents a skin pH of around 6.4, whereas chronic wounds have pH varying from 7.2 to 8.9. Considering the varied environmental pH levels to which the bacteria are subjected, the electrical excitability of S. epidermidis was explored across external pH (pHₑ) ranges of 4-9. The electrical stimulation condition (75 mVpp/μm, AC, 0.1 kHz for 10 s) was selected based on the work on B. subtilis stimulation by Stratford, J. P. et al. 31 and optimization processes outlined in FIG. 9.

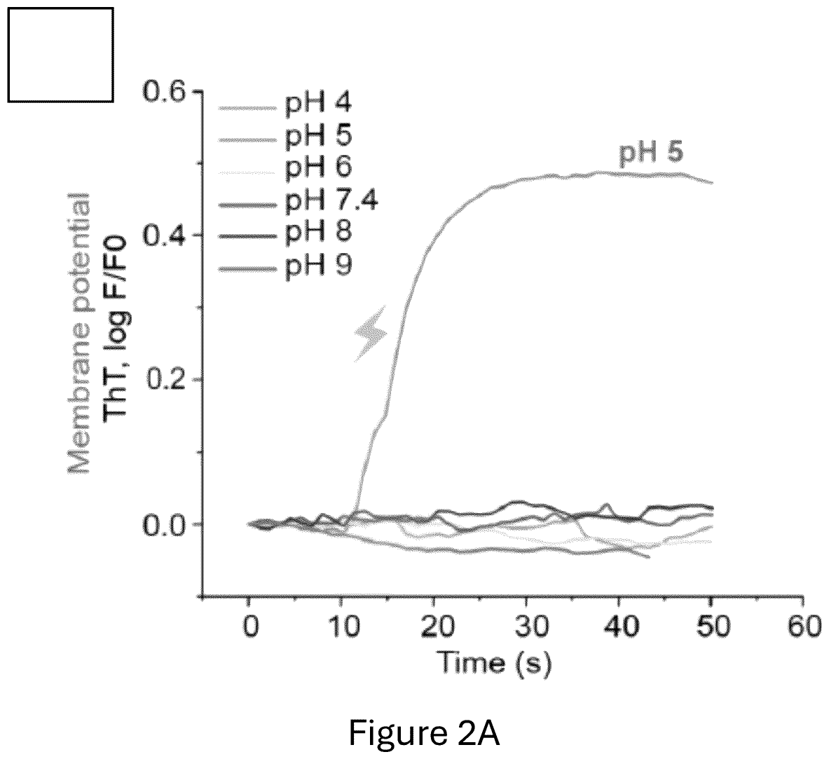

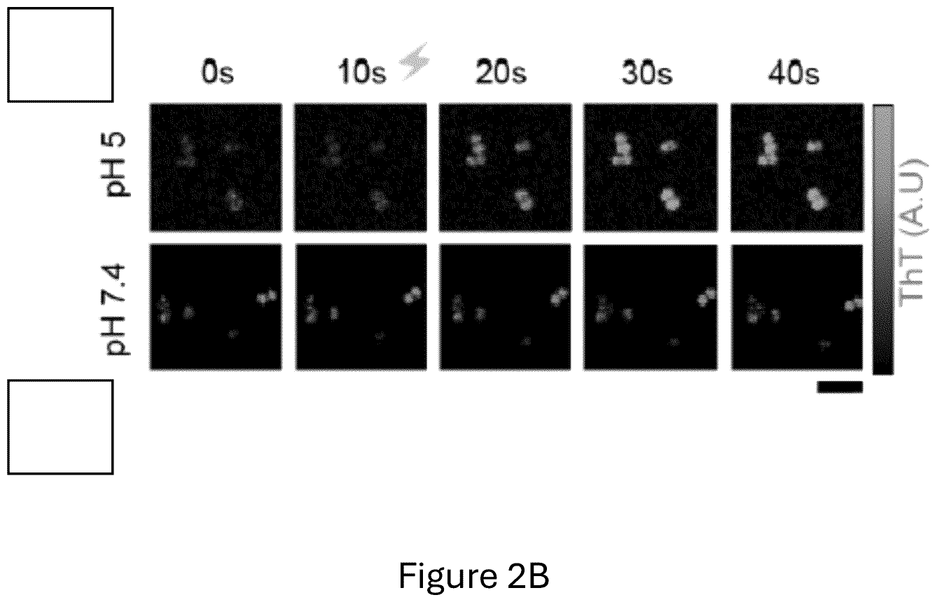

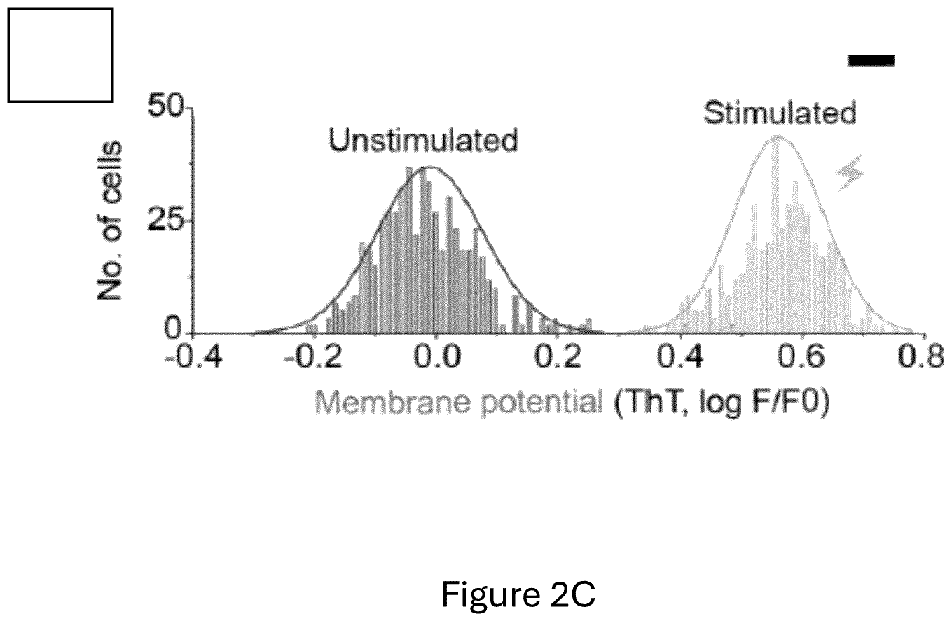

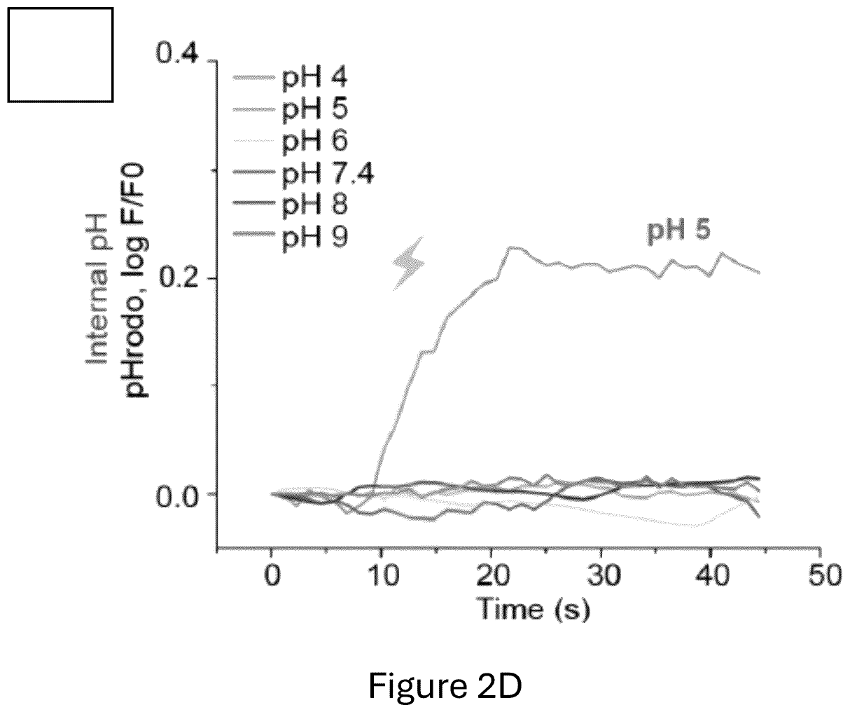

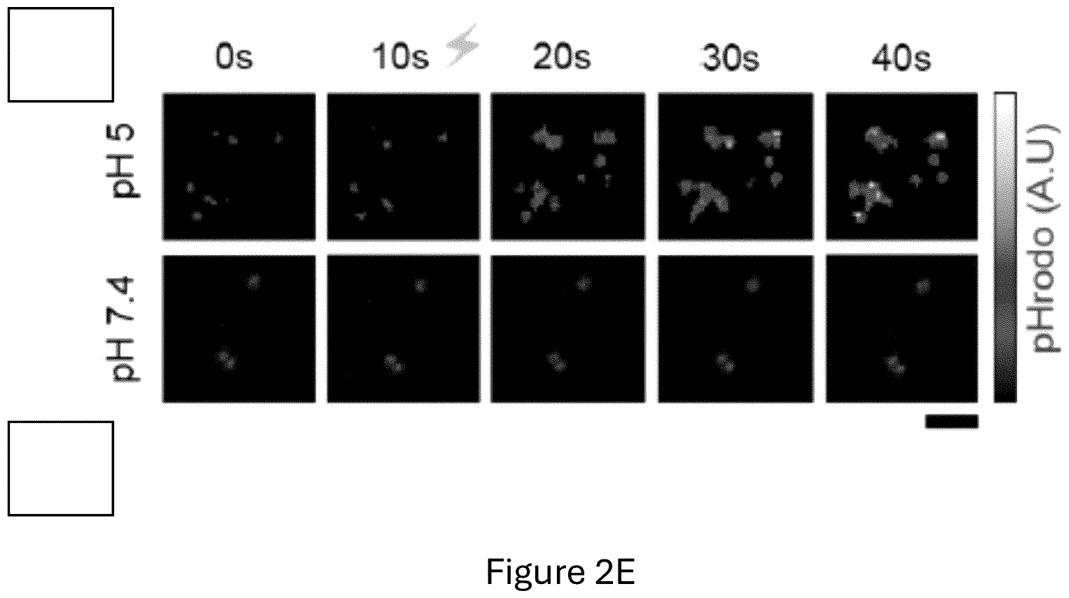

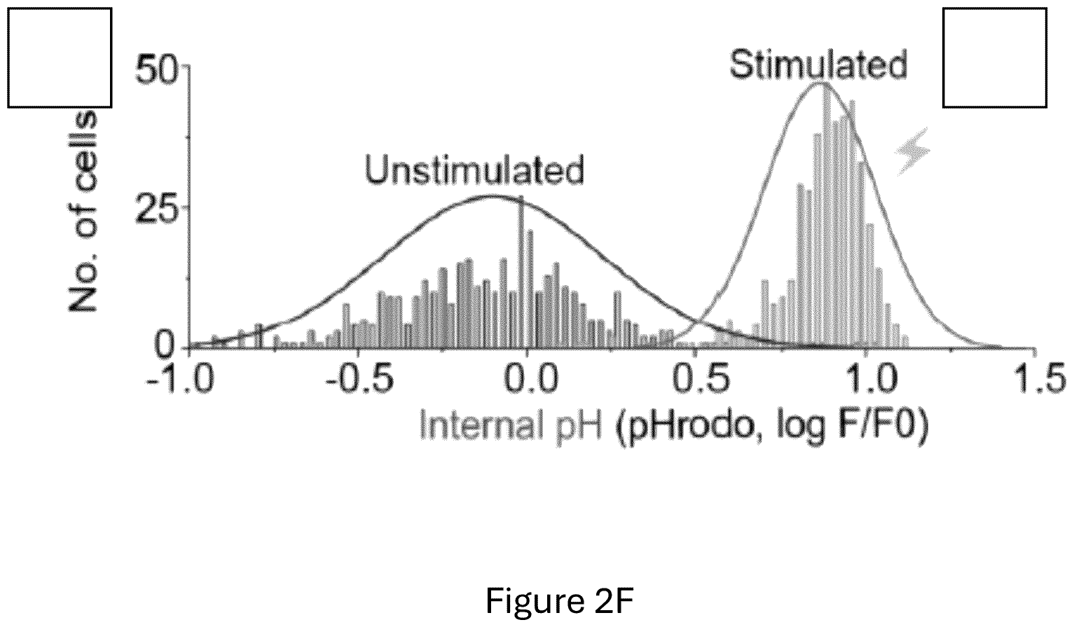

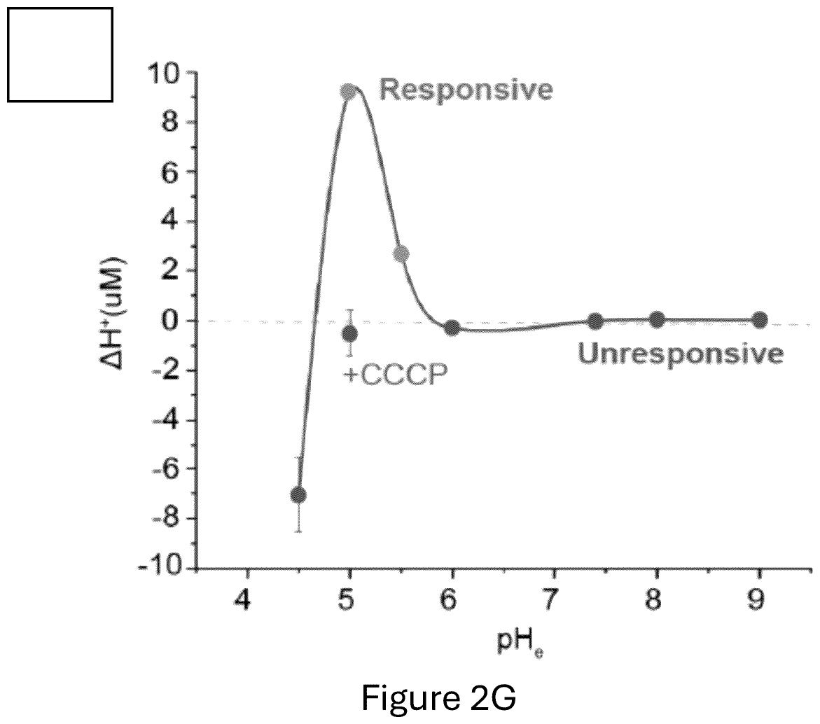

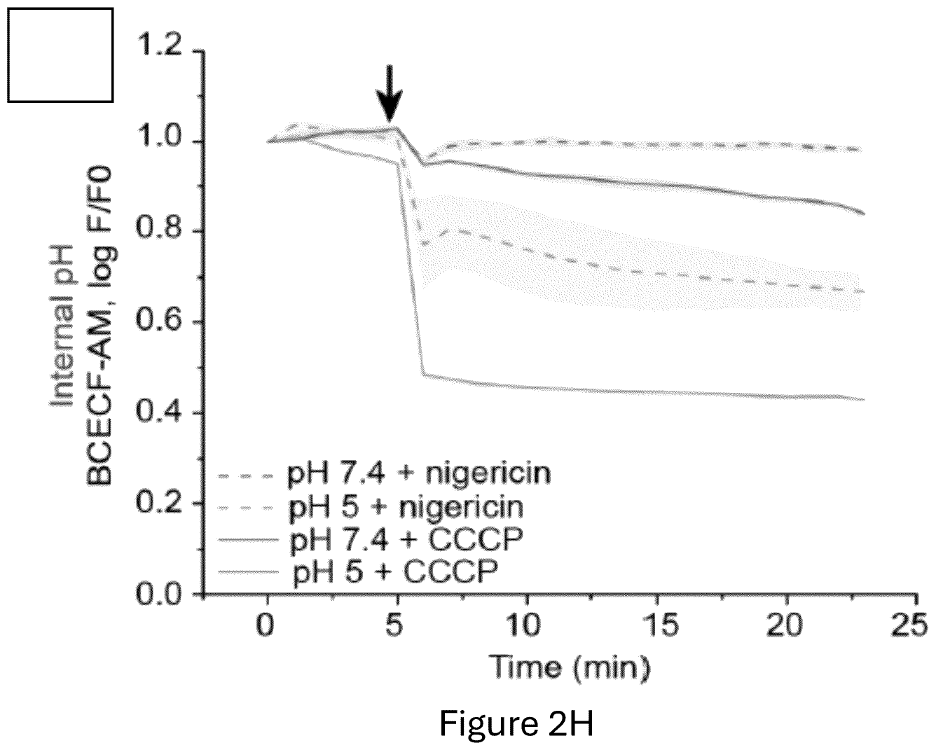

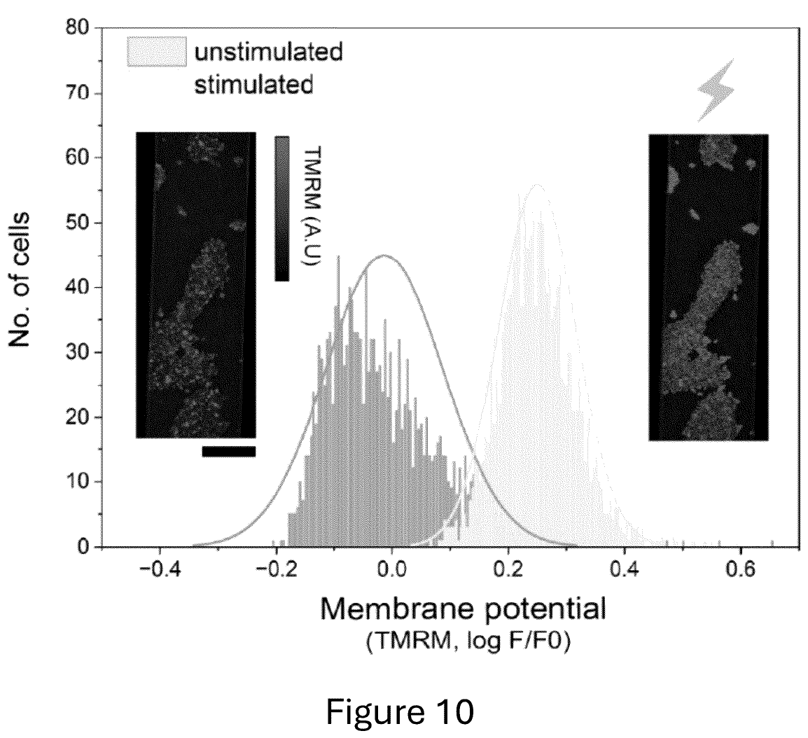

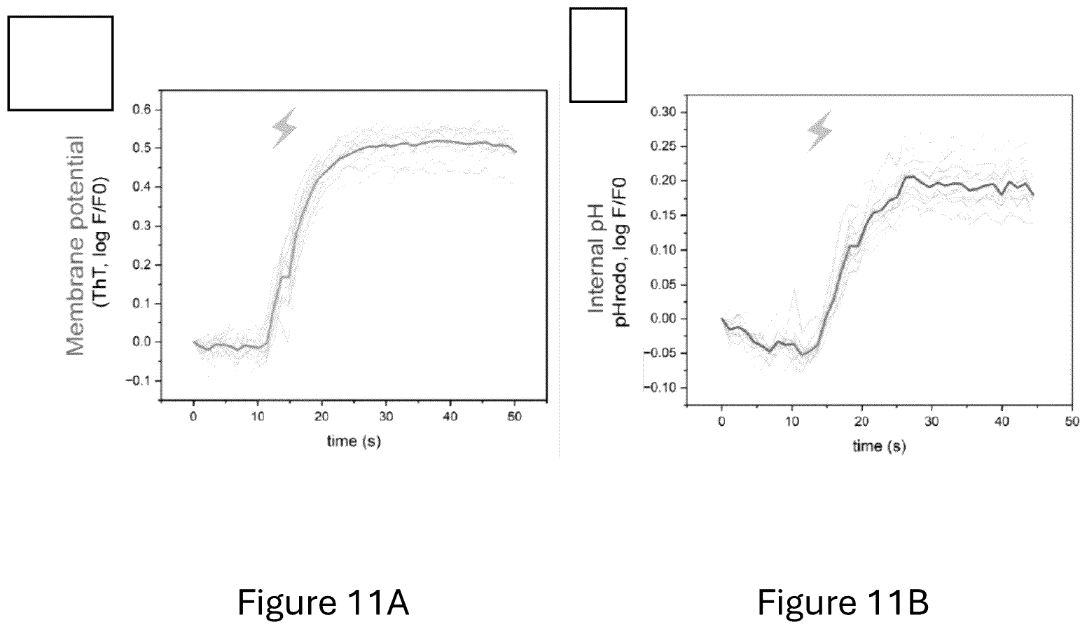

Electrical stimulation increased the fluorescence intensity of the membrane potential indicator Thioflavin T (ThT) in cells, indicative of hyperpolarization. However, this response was only observed at pHₑ=5, matching the pH of healthy skin (as shown in FIGS. 2A, 2B, 2C). Hyperpolarization of S. epidermidis upon electrical stimulation was confirmed with another membrane potential indicator, Tetramethylrhodamine (FIG. 10). Changes in the bacteria's intracellular pH (pHᵢ) using pHrodo staining was also studied. Electrical stimulation increased pHrodo fluorescence, suggesting cytoplasmic acidification. Like the membrane potential, the change was only observed at pHₑ=5 (as shown in FIGS. 2D, 2E, 2F). Single cell traces of ThT and pHrodo fluorescence showed that the excitatory response occurs across the population (FIG. 11).

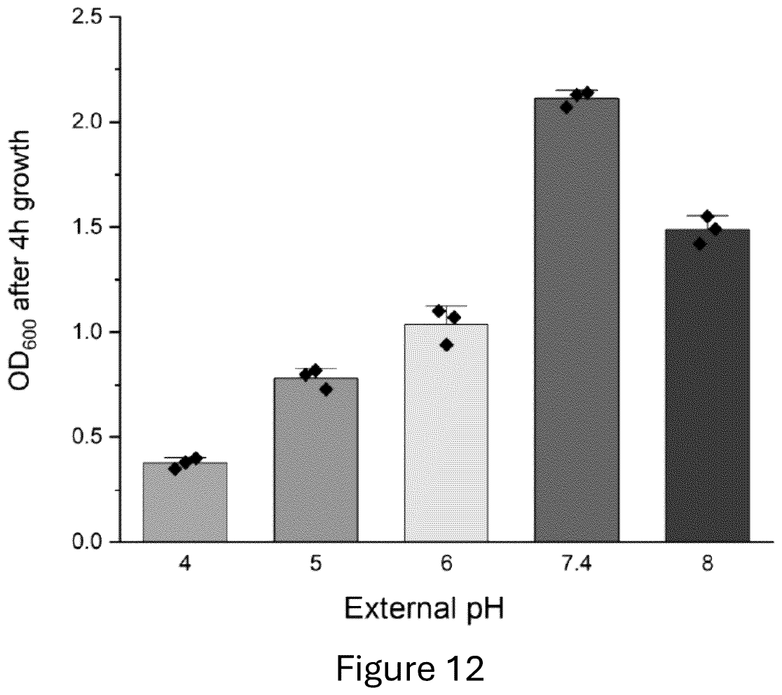

An acidic epidermal pH, used as skin's first line of defense against microbes, presents an adverse environment for bacterial growth. Surprisingly, S. epidermidis exhibited electrical excitability at the epidermal pH of 5 but remained completely unresponsive to stimulation at its optimal growth condition at pH 7.4 (FIG. 12). Herein, the term “selective excitability” is used to describe how the bacteria became excitable only when select conditions, deviating from the best thriving environment, were met.

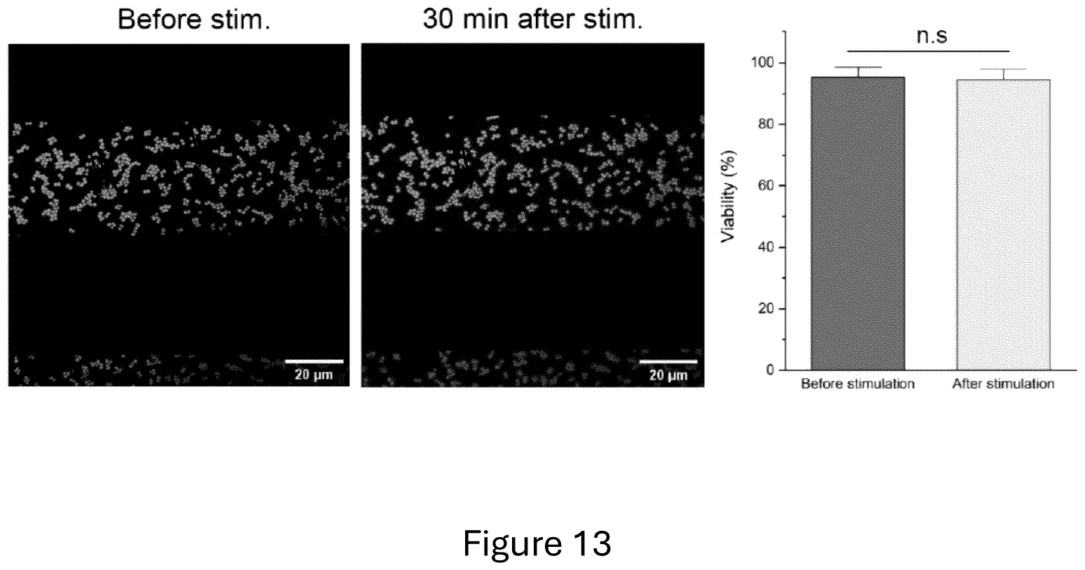

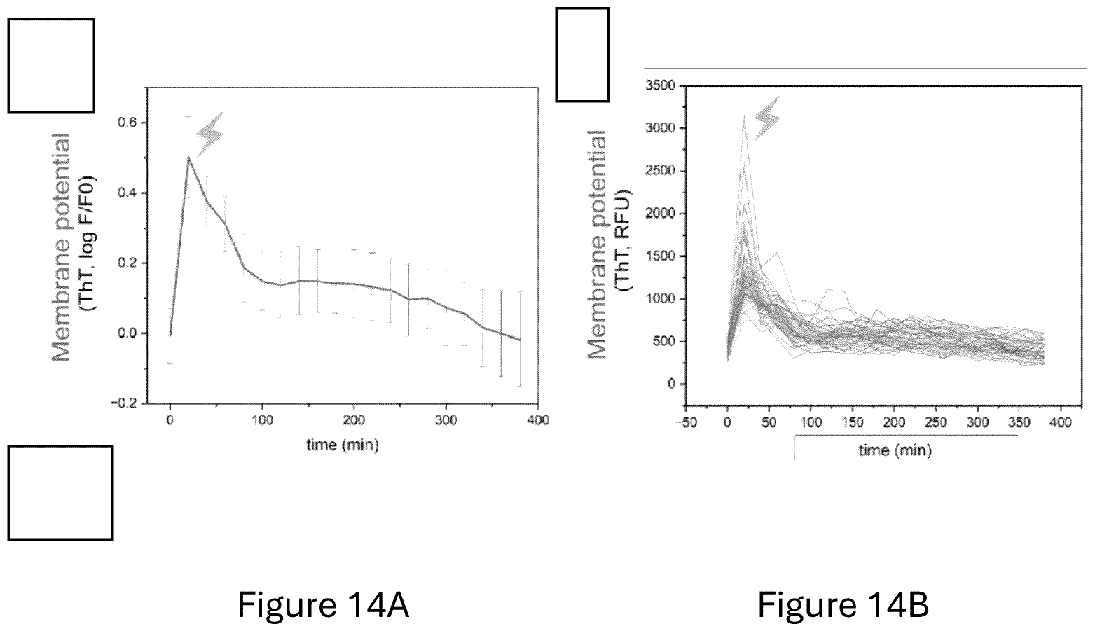

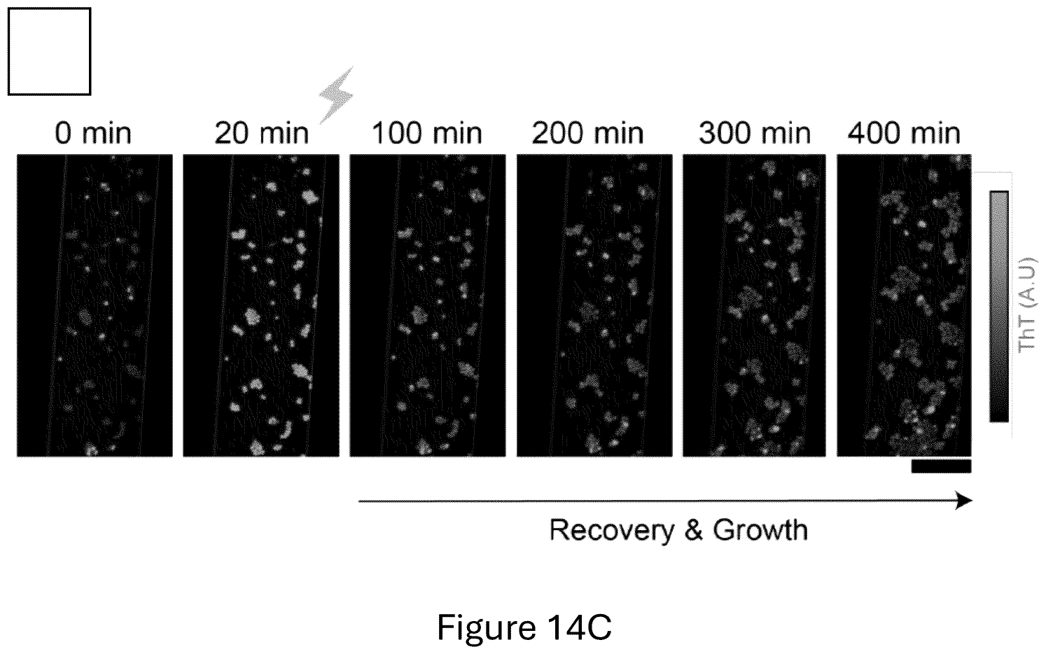

Next, several control experiments were conducted to demonstrate the reversibility and non-lethality of the excitation response. Changes in membrane potential and intracellular pH were not due to cell death or electroporation (FIG. 13), as the proportion of propidium iodide-stained cells remained unchanged upon a stimulation. Indeed, the electric field applied (75 mVpp/μm) was about two orders of magnitude lower than the lethal electroporation threshold for S. epidermidis reported in the literature, which ranges from 1000 mVpp/μm-3500 mVpp/μm. The electric field was generated with a voltage of 1.5 V_{ac}, which is considered safe and imperceptible to humans. Most importantly, the hyperpolarized membrane potential could be reversed back to the resting state within a 6-hour timeframe, during which the bacteria resumed growth (FIG. 14). These results show the reversibility and non-lethal nature of the bioelectrical response.

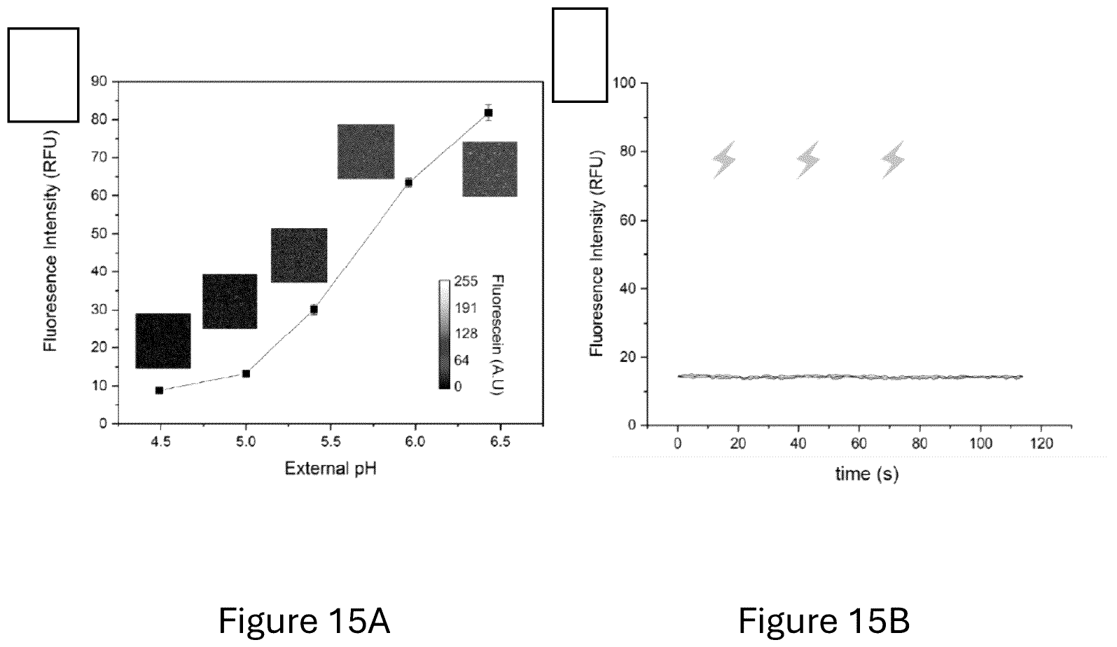

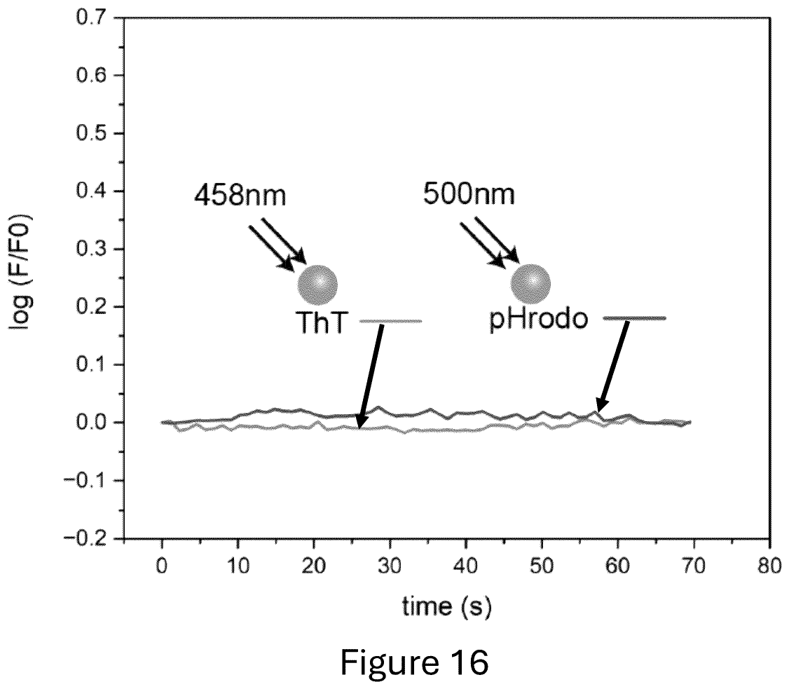

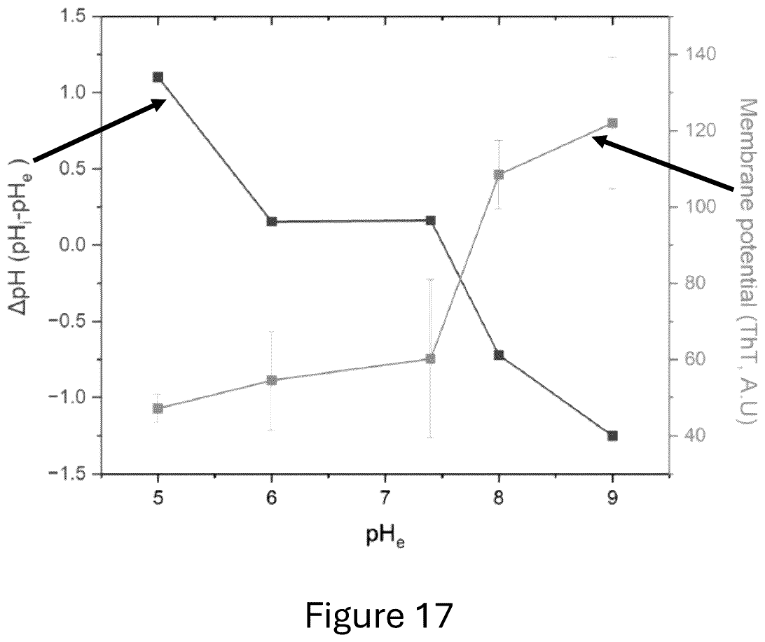

More control experiments were conducted to understand the nature of the excitatory response. Using fluorescein as a pH probe, it was verified that external pH, which is known to affect membrane potential, remains unaltered during and after the stimulation (FIG. 15). Further, it was found that the excitation laser for ThT and pHrodo alone does not trigger an increase in fluorescence (FIG. 16). The simultaneous rise in ThT and pHrodo fluorescence during electrical stimulation aligned with reported connections between internal pH and membrane potential. Specifically, it is known that the decrease in internal pH is balanced by hyperpolarization of the membrane potential as part of bacterial pH homeostasis (see FIG. 17 for details). Overall, the experiments suggested that the observed phenomenon is an authentic electrophysiological response elicited by electrical stimulation.

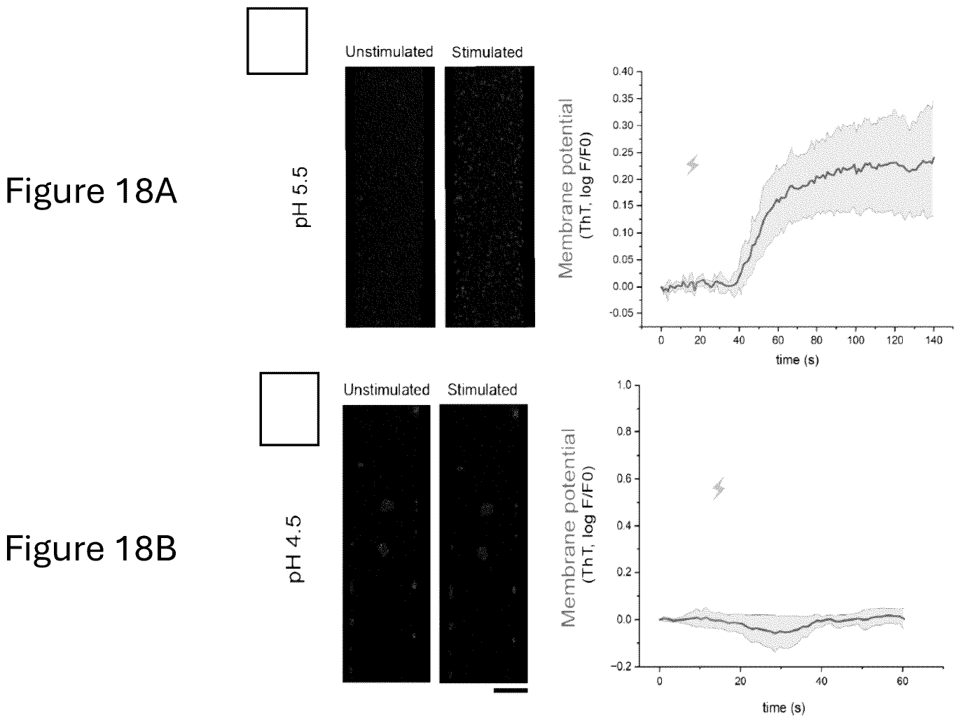

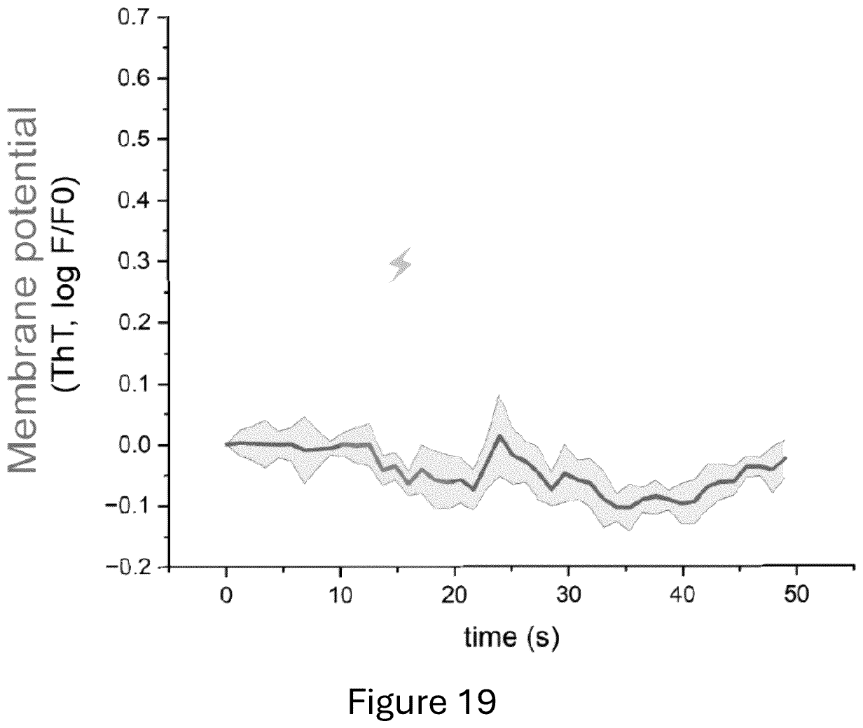

Next, it was investigated why bacteria only responded at pH 5 and how the epidermal pH confers excitability to S. epidermidis. By using the intracellular pH dye (BCECF-AM), it was found that the transmembrane proton gradient, ΔH⁺, is small at pHₑ≥6, and collapses at acidic pHₑ≤4.5. The ΔH⁺, however, strongly peaked at pHₑ=5 (FIG. 2G). Since electrical excitation of S. epidermidis involves proton influx, the large ΔH⁺ near epidermal pH can be correlated with a greater driving force for the electrical response. Indeed, it was observed that S. epidermidis can be excited only when significant ΔH⁺ is present, at pH 5 and 5.5 (FIG. 18). Conversely, abolishing the proton gradient by adding the protonophore CCCP completely inhibited the excitation response (FIG. 19). The results indicated that the presence of ΔH⁺ is critical for the excitability of S. epidermidis.

To demonstrate how large ΔH⁺ drives proton influx when cells are perturbed, kinetic measurements of BCECF-AM fluorescence (FIG. 2H) were conducted. Upon adding CCCP and nigericin, two ionophores known to induce proton influx, there is a significant drop in BCECFAM fluorescence, indicative of cytoplasmic acidification, at pHₑ=5. At pHₑ=7.4 however, the change is less pronounced. The differences were expected as ΔH⁺ at pHₑ=5 is orders of magnitude larger than at pHₑ=7.4 (FIG. 2G). Altogether, the result suggests that selective excitability at the epidermal pH range of 5-5.5 arises due to a large ΔH⁺ gradient, which provides driving force for proton influx when perturbations, such as electrical stimulation, are applied.

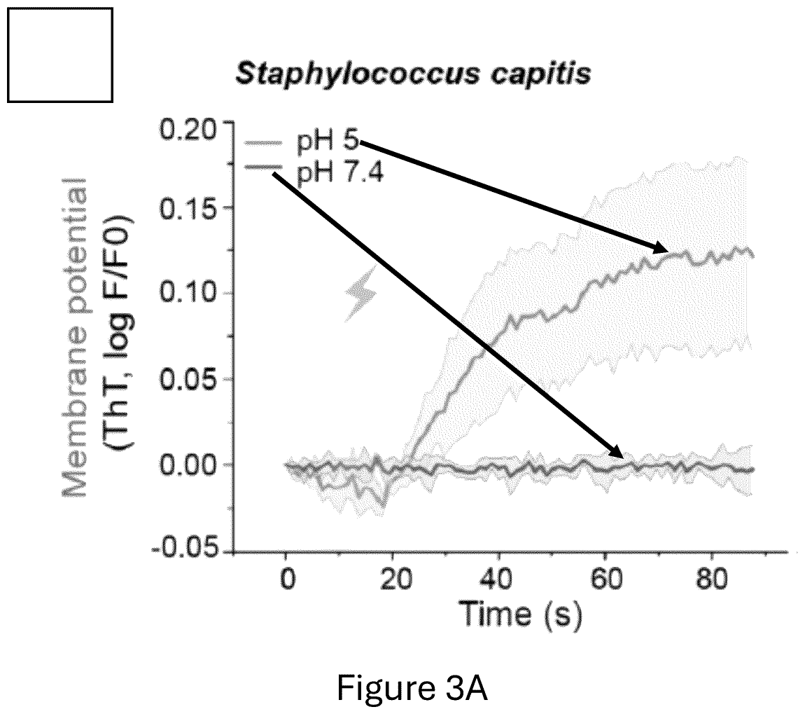

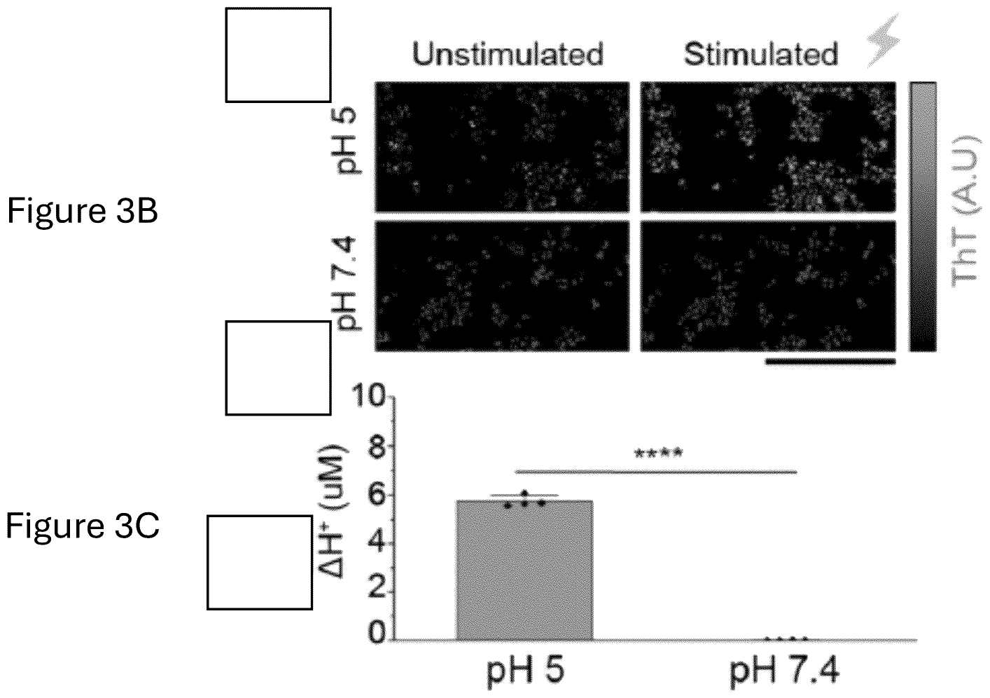

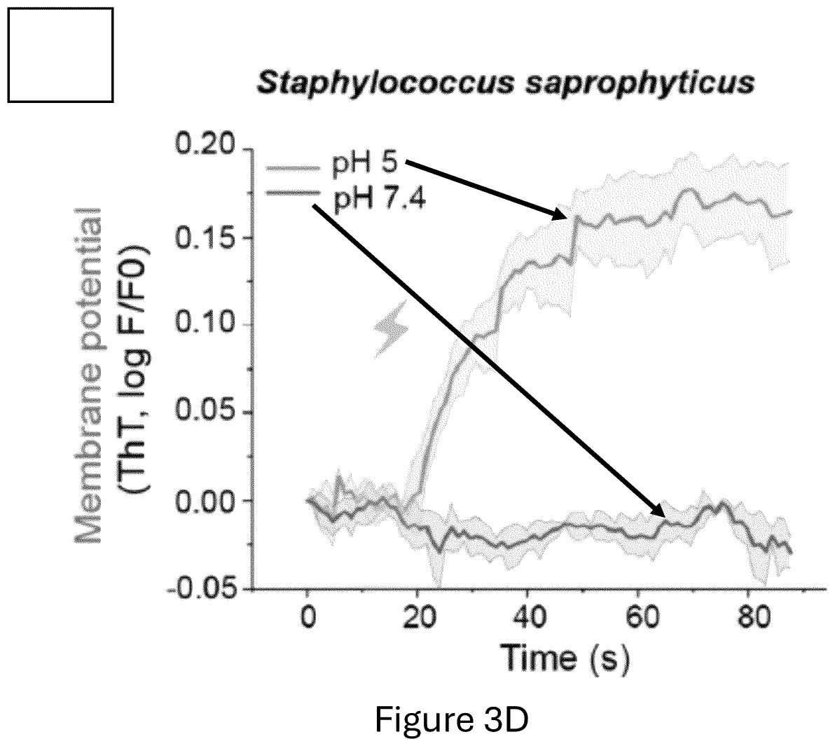

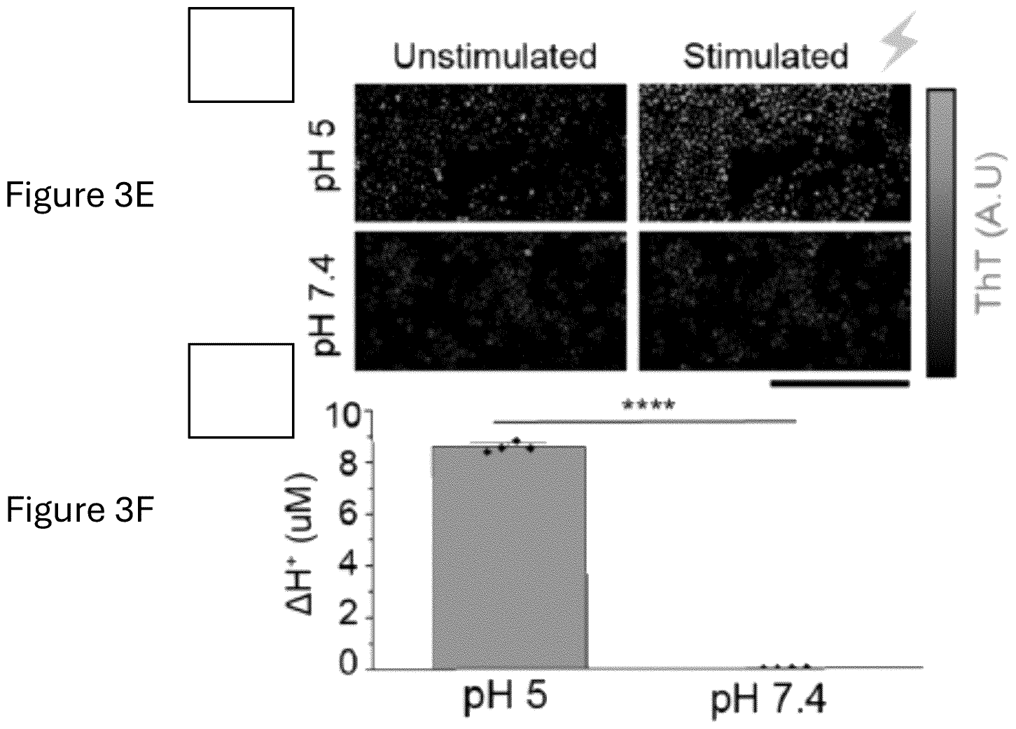

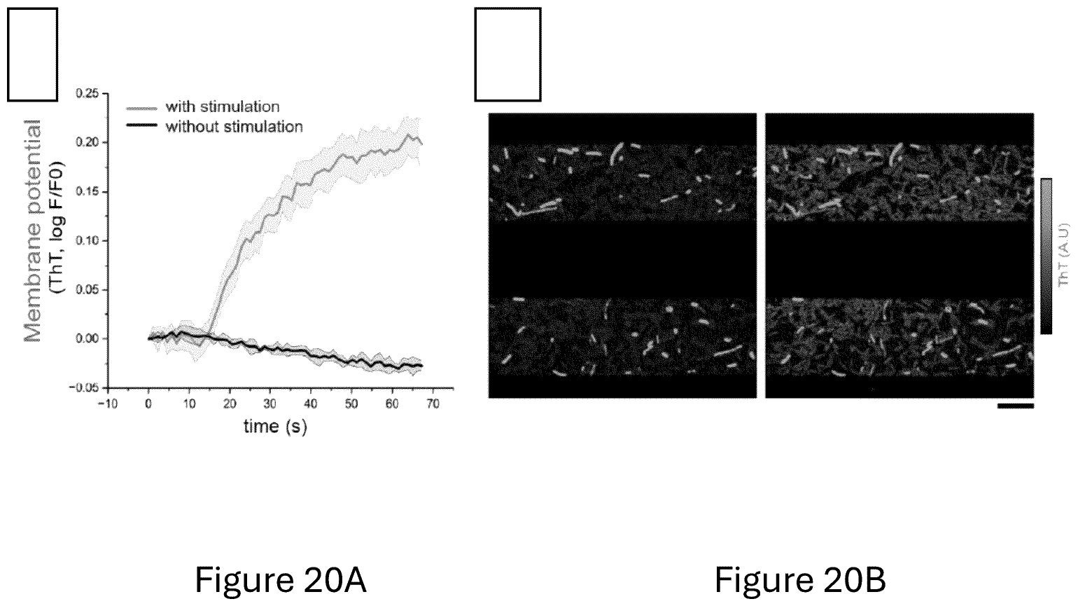

Interestingly, it was also noted that epidermal pH confers electrical excitability to other opportunistic pathogens-Staphylococcus capitis and Staphylococcus saprophyticus. S. capitis, part of the normal skin flora, causes opportunistic infections in prosthetic medical devices and neonatal sepsis. S. saprophyticus, prevalent in the acidic environment of the skin, vagina, and urogenital tract, is responsible for 10-20% of urinary tract infection in sexually active young women worldwide. Upon electrical stimulation, S. capitis displays hyperpolarization at pHₑ=5 but not at pHₑ=7.4. (FIGS. 3A, 3B). Similarly to S. epidermidis, the driving force for proton influx, ΔH⁺, was two orders of magnitude larger at pHₑ=5 than at pHₑ=7.4 (FIG. 3C). Identical trends were observed for S. saprophyticus (FIGS. 3D, 3E, 3F). The results demonstrate the existence of selective excitability in the skin-residing microbes that were previously not known to be excitable. While exploring trends in the electrical response of resident bacteria is beyond the scope, future investigation into this topic will help determine how selectively electrical stimulation can target bacteria on skin (FIG. 20).

Programmable Electrical Stimulation Suppresses Virulence Factors in S. epidermidis

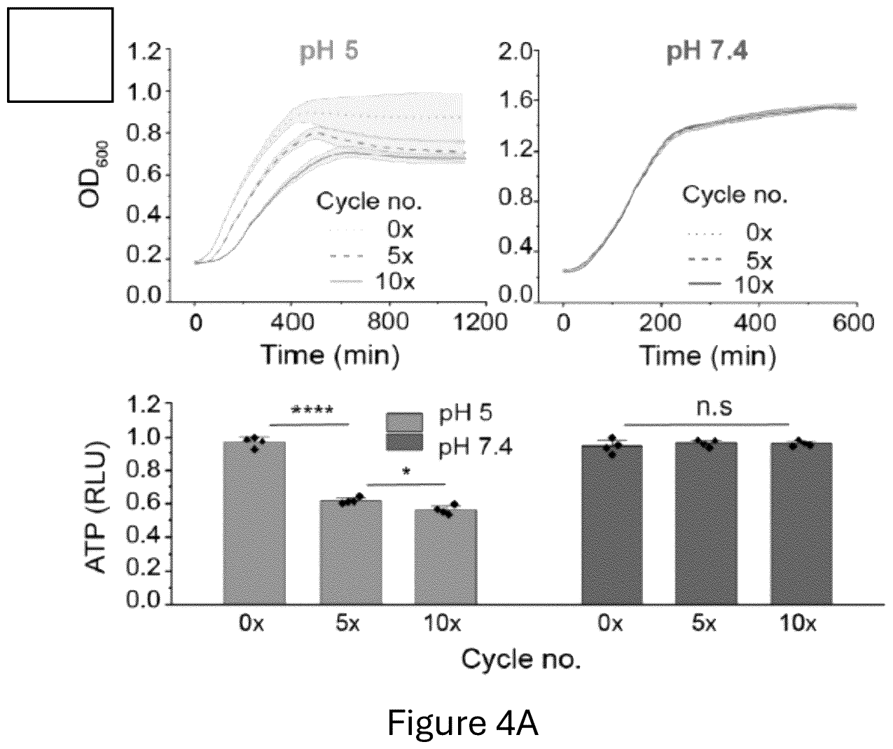

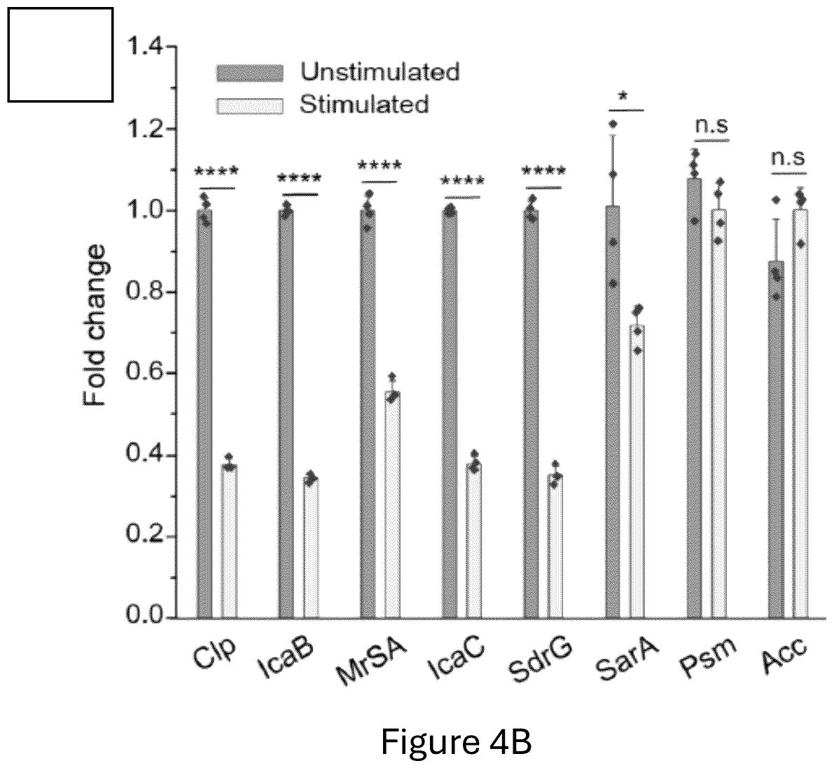

The next aim was to understand the long-term effects of electrical stimulation on S. epidermidis at a population level. It was observed that electrical stimulation reduces both growth and ATP levels in S. epidermidis with successive stimulation cycles (FIG. 4A). This was expected, since perturbations in membrane potential and intracellular pH have been reported to interfere with normal cellular functions, including proliferation and respiration. The reduction was not observed at pHₑ=7.4 where no excitation response was observed. Moreover, transcriptional analysis post-electrical stimulation revealed a decrease in the expression of virulence genes responsible for surface adhesion (SdrG), polysaccharide intracellular adhesin (icaB, icaC), protease (Clp), antibiotic resistance (MrSA), and virulence regulation (SarA) (FIG. 4B). The results show that the excitatory response accompanies a suppressive effect on the virulence of the opportunistic pathogen.

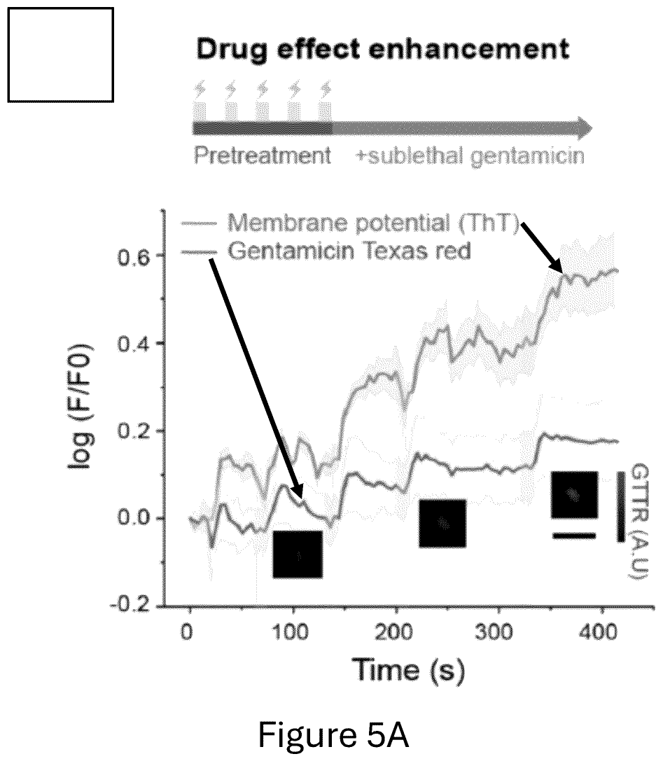

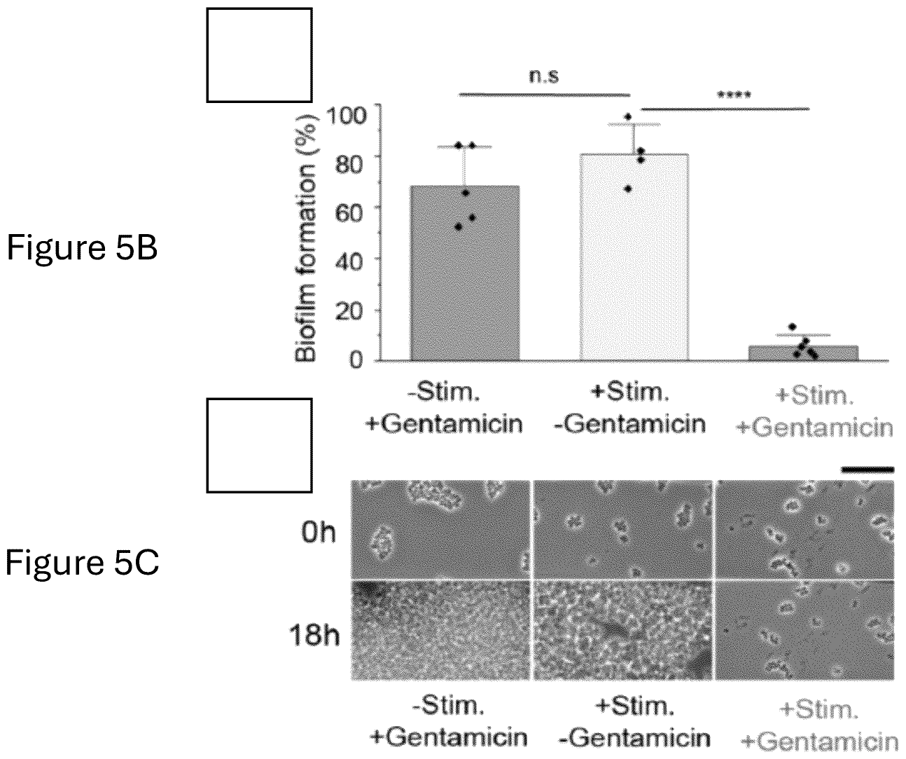

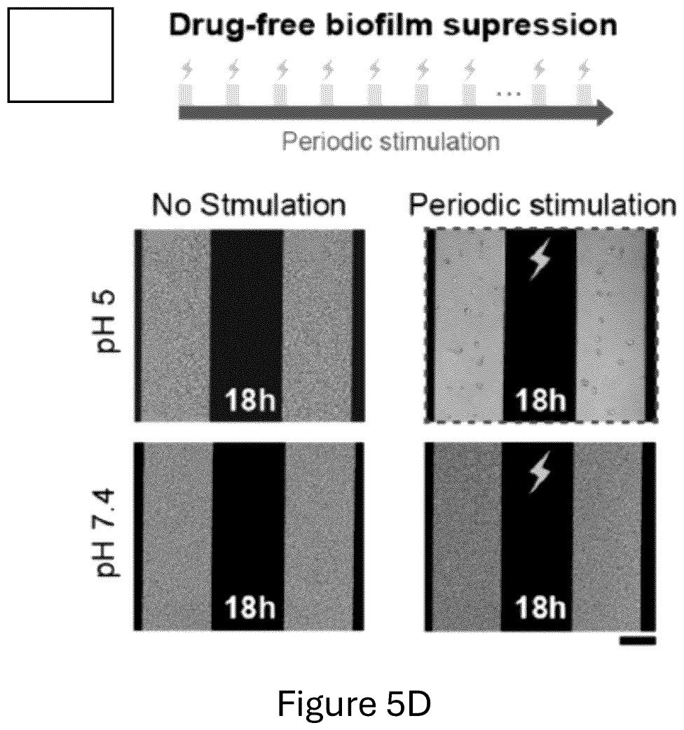

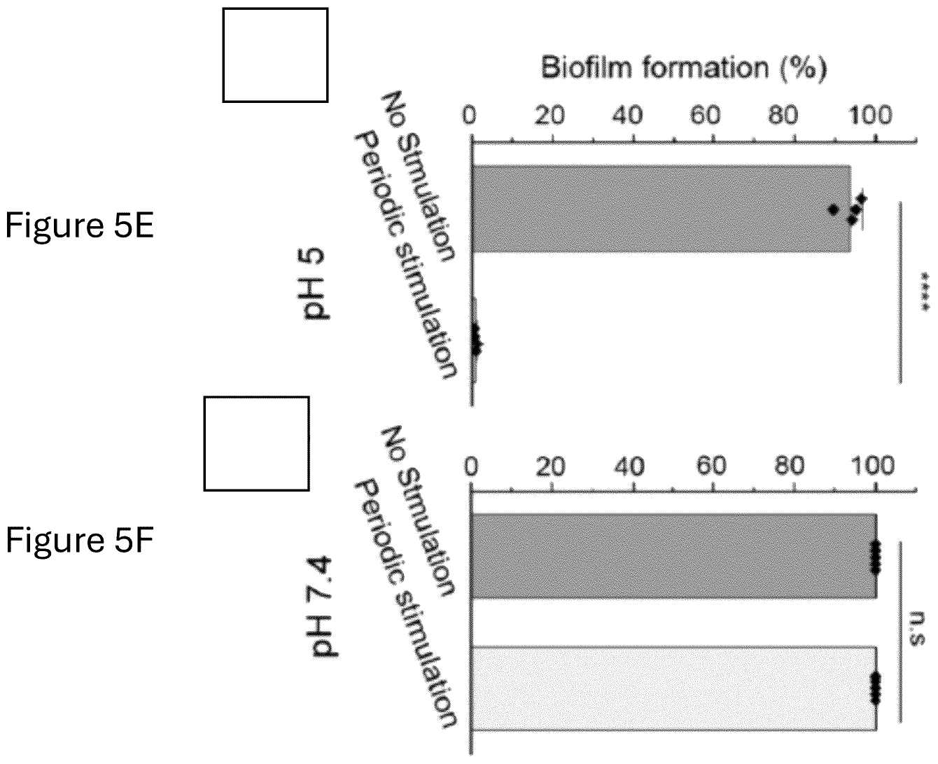

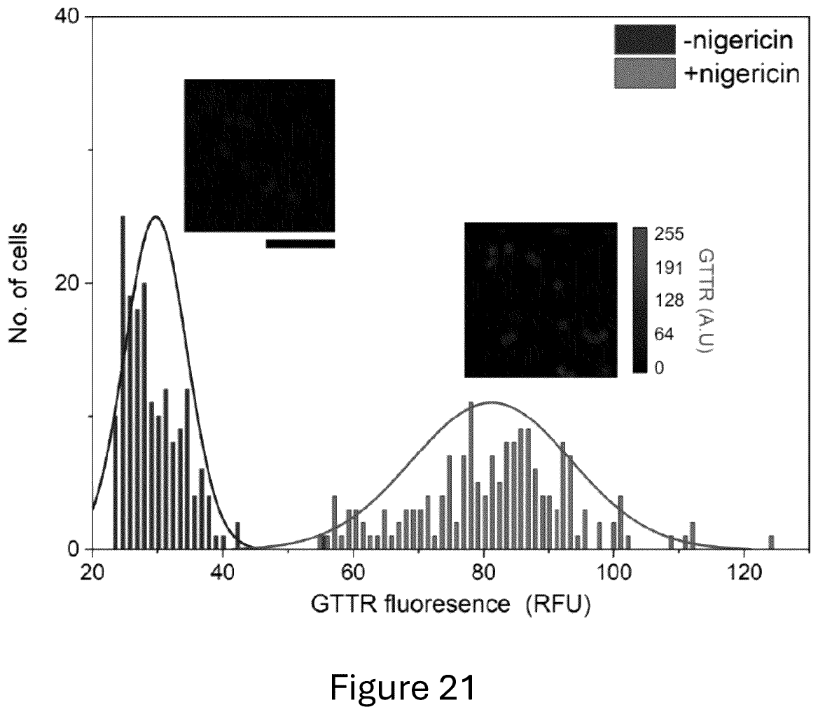

It was further investigated whether electrical stimulation could assist in controlling biofilm formation, the defining virulence factor in S. epidermidis. Given that electrical stimulation hyperpolarizes S. epidermidis, making the cells more negatively charged, it was hypothesized that this would increase the cell's vulnerability to positively charged aminoglycoside antibiotics such as gentamicin (FIG. 21). Indeed, co-staining S. epidermidis with ThT and Gentamicin-Texas Red (GTTR) shows that repeated stimulation leads to successive hyperpolarization and accumulation of gentamicin (FIG. 5A). Hence, a protocol was programmed to enhance the inhibitory effect of drug through electrical pretreatment. The protocol involved 5 cycles of stimulation pretreatment to induce hyperpolarization, followed by incubation with a sublethal dose of gentamicin for 18 hours. While electrical pretreatment or a sublethal dosage of gentamicin alone failed to reduce biofilm coverage by more than 30%, their combined application resulted in a 94% decrease in biofilm coverage (FIG. 5B, 5C). This demonstrates that electrical stimulation can enhance the effect of the drug, enabling biofilm inhibition with reduced dosages of antibiotics.

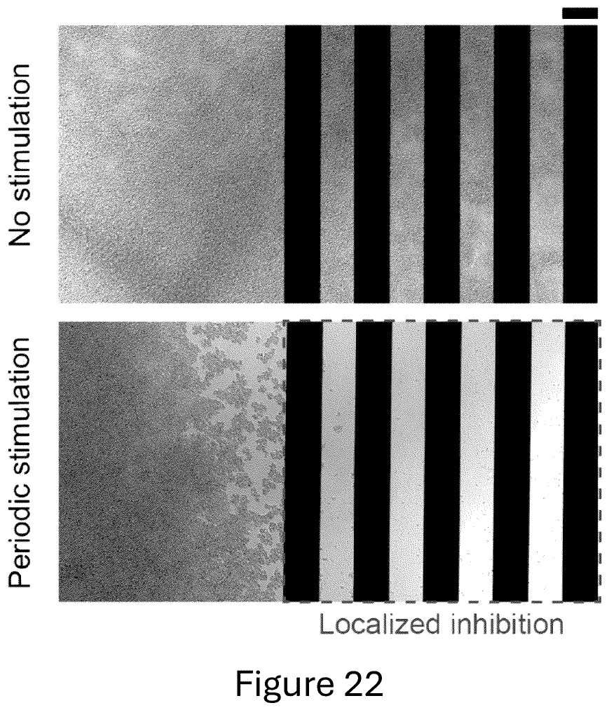

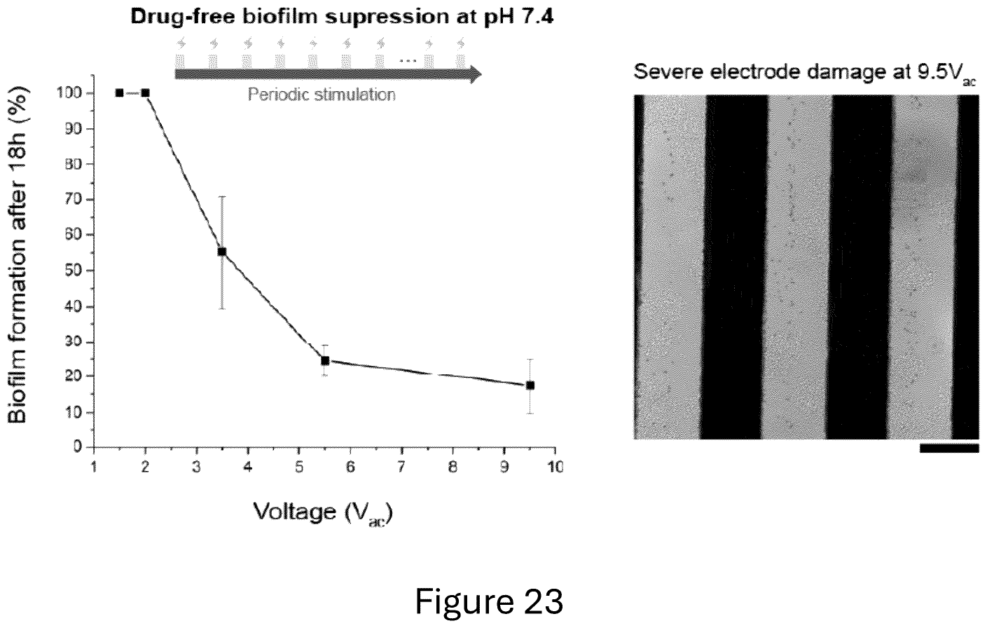

Next, the possibility of controlling biofilm formation solely through electrical means, without the use of drugs, was investigated. As mentioned earlier, S. epidermidis can recover its membrane potential and resume growth upon stimulation (FIG. 12). Thus, a protocol was developed which applies electrical stimulation at periodic intervals, every 10 minutes, to prevent the recovery and achieve long-term suppression. The periodic stimulation at pHₑ=5 eradicated biofilm formation by 99% without the need for antibiotics (FIGS. 5D, 5E). The effect of inhibition was localized where the bulk interdigitated electrode arrays were present (FIG. 22). No reduction in biofilm formation was observed at pHₑ=7.4 (FIG. 5F) where excitation response was not observed, indicating that the periodic electrical stimulation itself is non-lethal for the bacteria. In the absence of an excitatory response at pH 7.4, a voltage as high as 9.5 V_{ac} was required to achieve a similar, albeit less effective, level of biofilm suppression using the identical protocol (FIG. 23). The low-voltage and drug-free electrical suppression of biofilm growth was uniquely enabled at the epidermal pH where S. epidermidis displays selective excitability.

### IV. Bioelectronic Localized Antimicrobial Stimulation Therapy (Blast) Harnessing Selective Excitability

Chronic skin inflammation and wounds are linked with elevated pH levels and colonization of virulent S. epidermidis that can exacerbate the conditions. To address the issue, an electroceutical device was developed that can restore the skin's acidic pH, sensitizing S. epidermidis to electrical stimulation and suppression. The device was used to deliver Bioelectronic Localized Antimicrobial Stimulation Therapy (BLAST), which controls proliferation of the opportunistic pathogen through a drug-free bioelectrical stimulation. The stimulation parameters for BLAST were set identically to the drug-free suppression protocol in FIG. 5D.

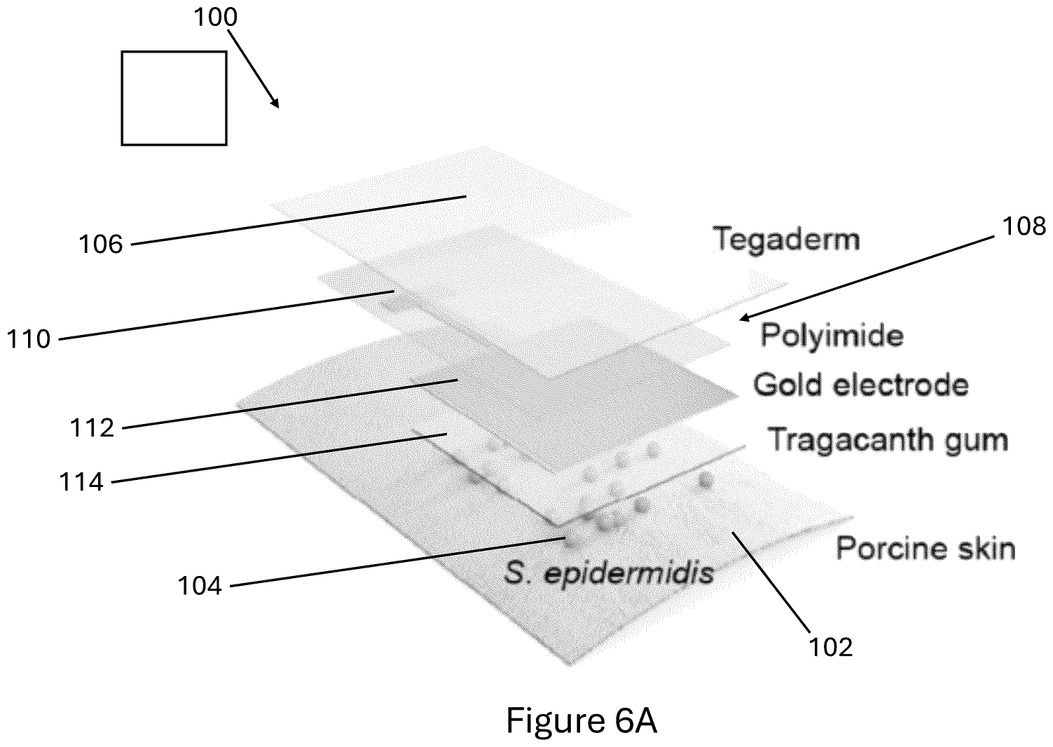

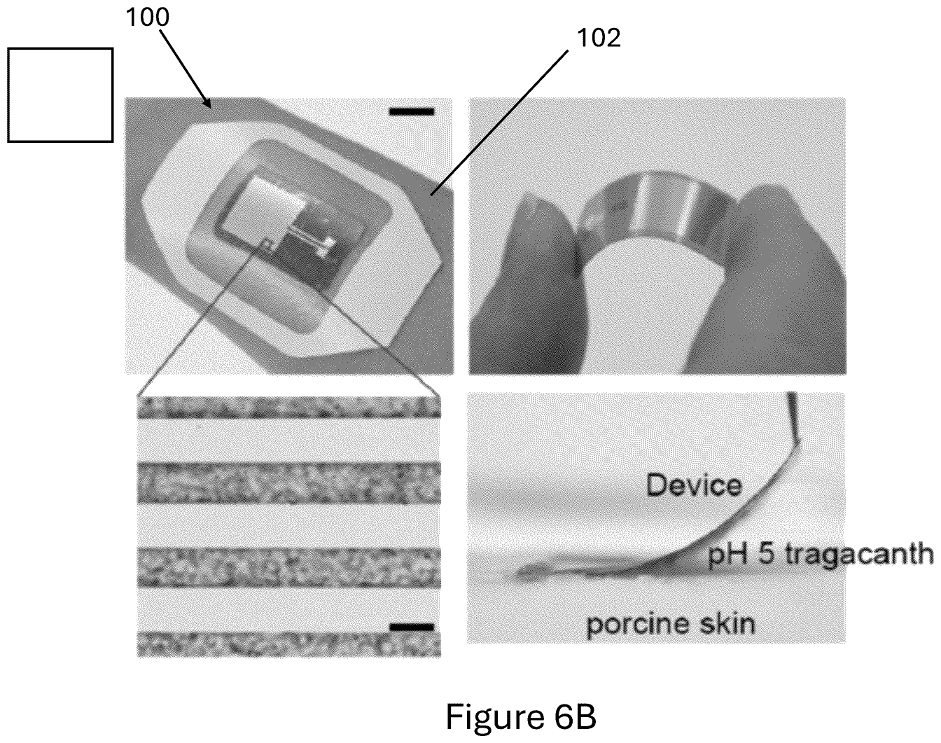

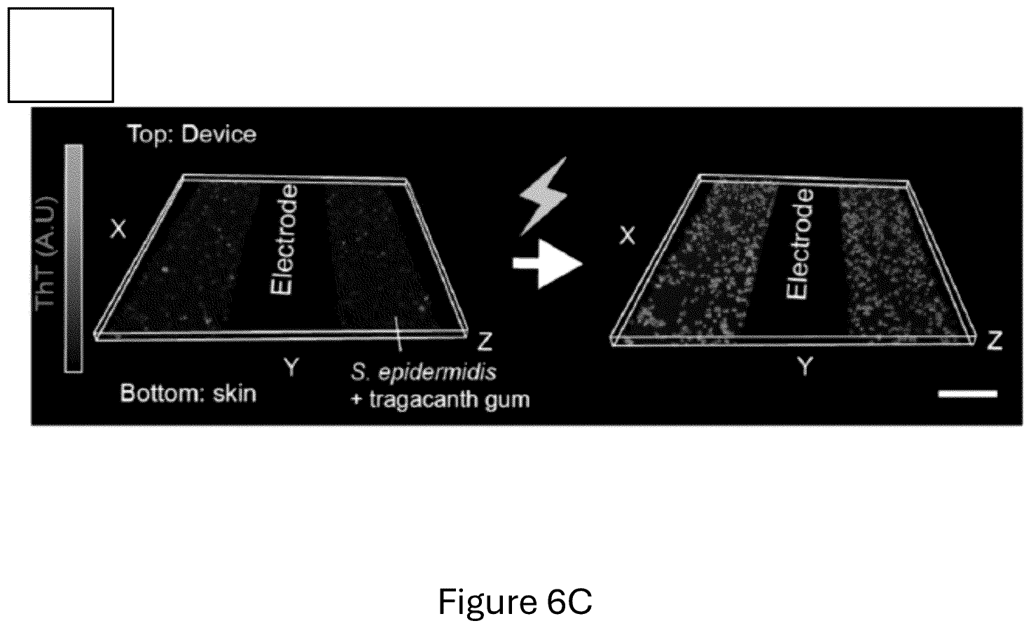

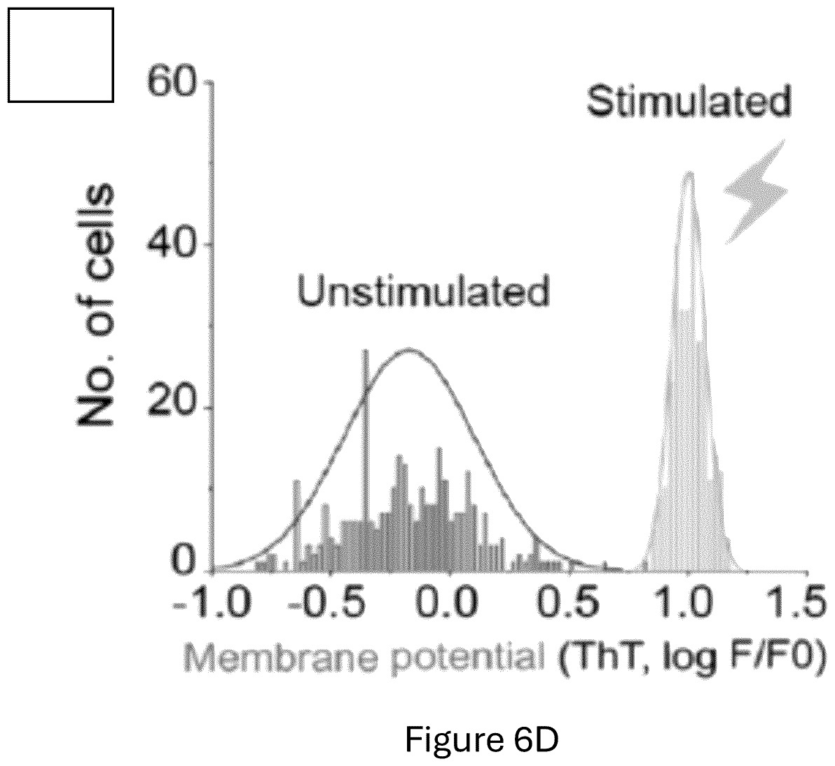

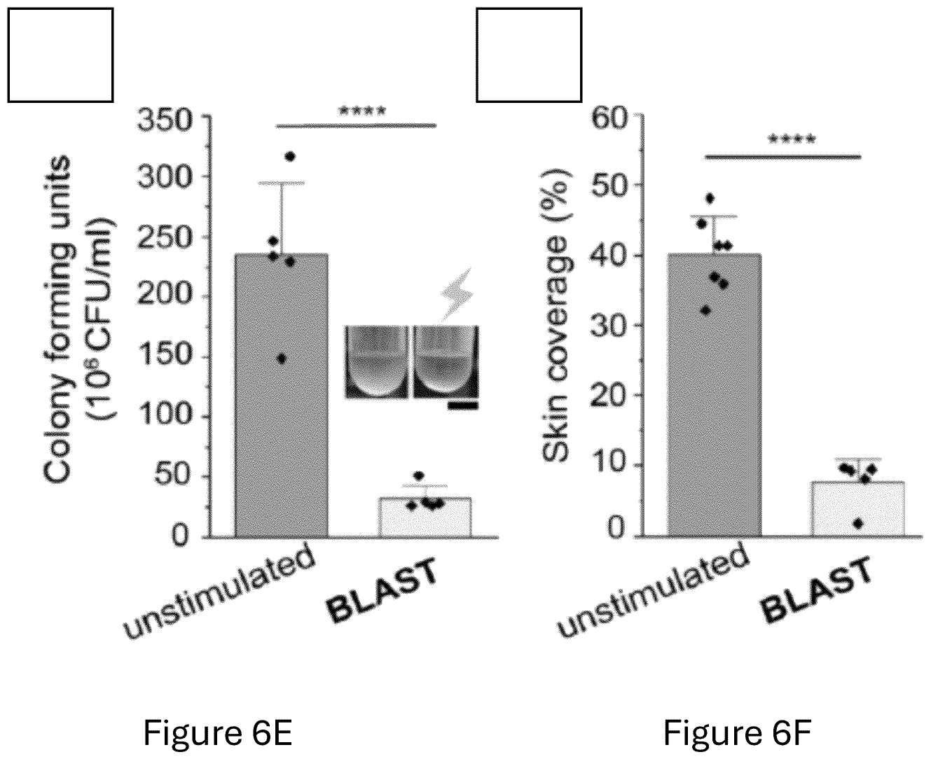

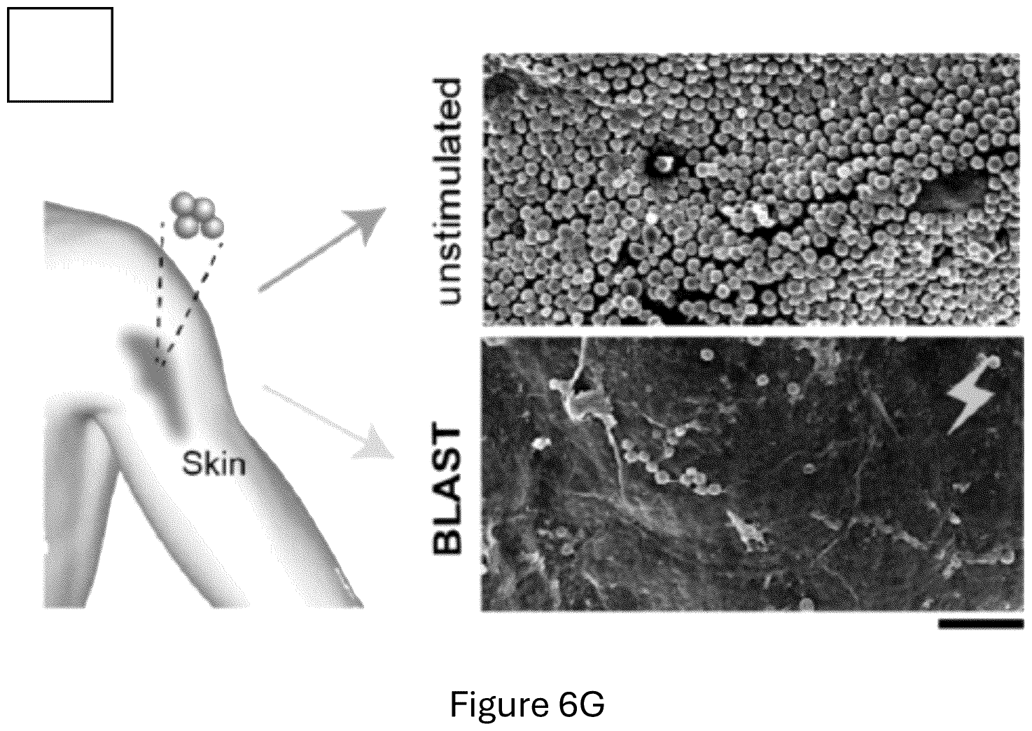

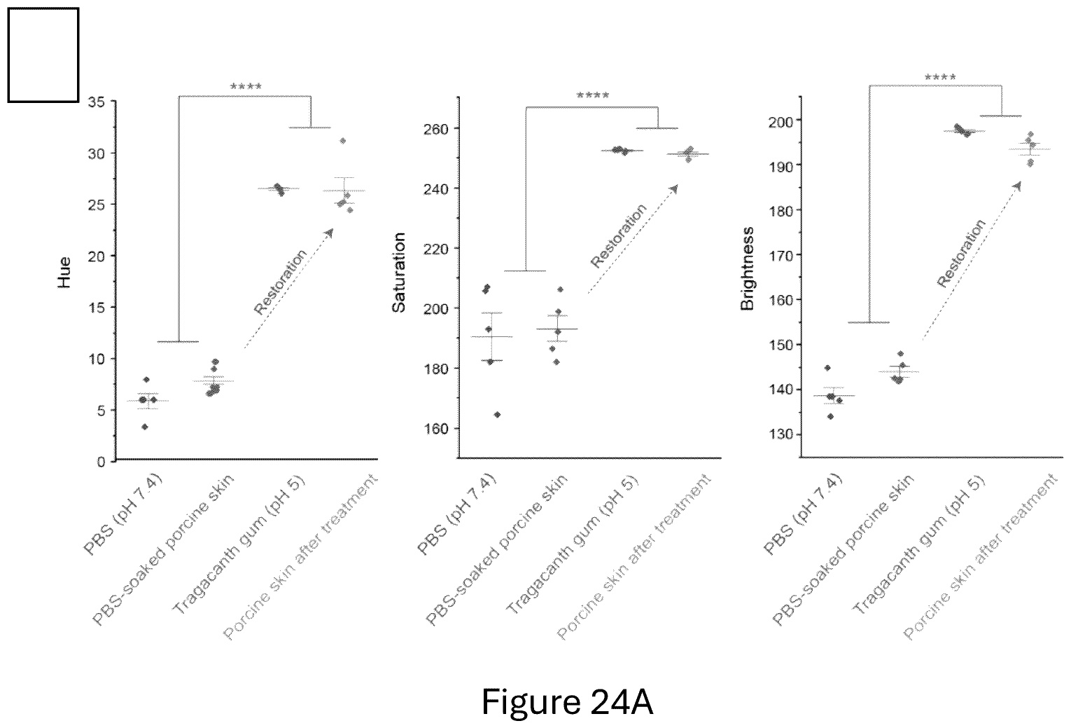

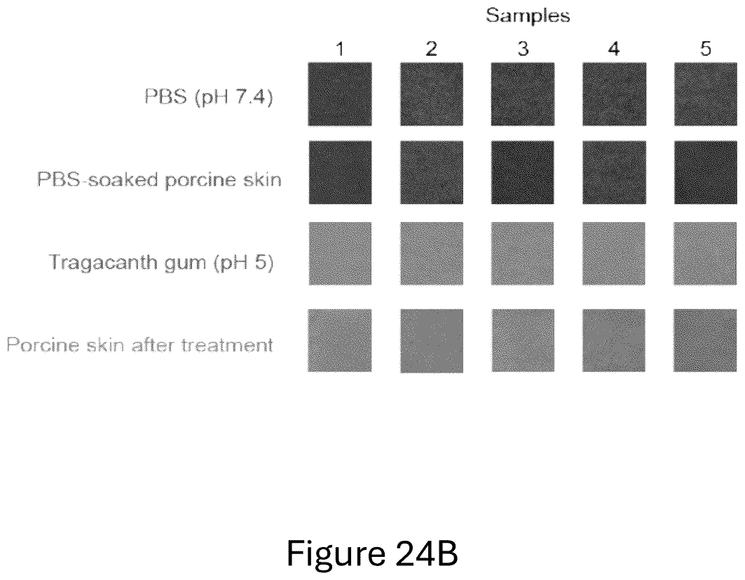

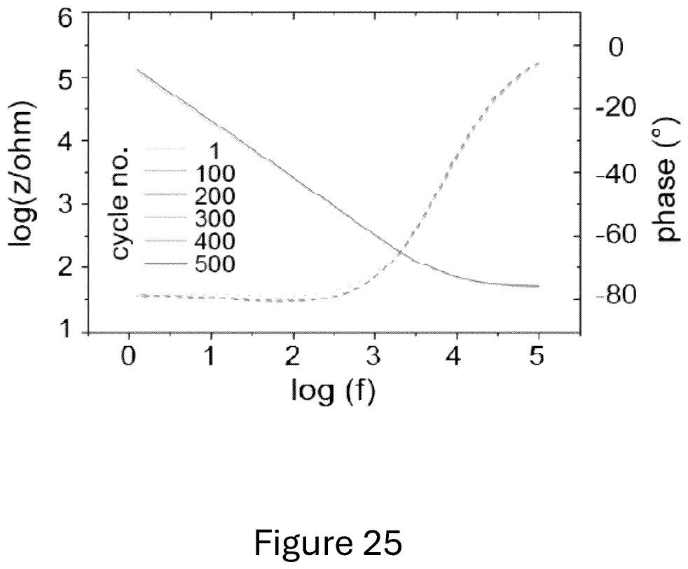

The electroceutical device featured an interdigitated electrode array on a flexible polyimide substrate (FIG. 6A, 6B). pH 5-adjusted tragacanth gum served as a hydrogel interlayer between the skin and device, which provided acidic environment that confers excitability to S. epidermidis (FIG. 24). Tragacanth gum was selected for its biocompatibility and its natural ability to form an acidic pH upon gelation. With 500 cycles of periodic stimulation lasting 5 days, the device showed no significant decline in impedance, indicating its stability (FIG. 25). The stimulation condition was benign for humans; a voltage was utilized of 1.5V_{ac}, which is an order of magnitude below the most conservative 15V_{ac} voltage limit deemed imperceptible and safe for wet contact. When the device was applied to a surface inoculated with S. epidermidis, electrical stimulation elicited the hyperpolarization response as shown by confocal z-stack imaging (FIGS. 6C, 6D). This confirmed that the device can stimulate S. epidermidis on porcine skin.

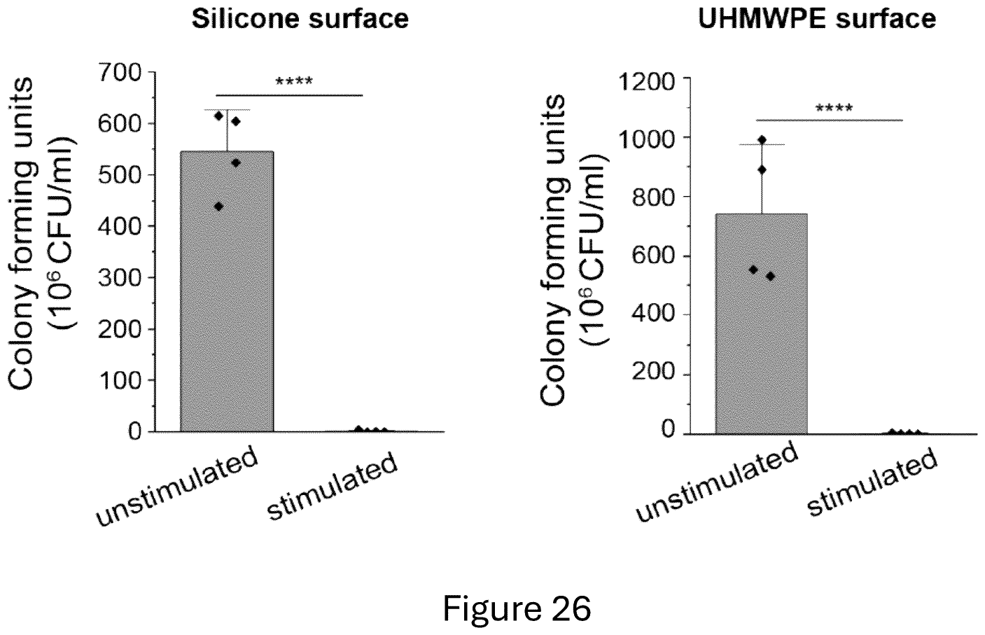

To showcase the potential of BLAST in controlling opportunistic pathogens, the device with was interfaced with porcine skin inoculated with S. epidermidis. Porcine skin was selected for its similarity to human skin. After 18 h of BLAST treatment, a nearly 10-fold reduction in the colony-forming units on porcine skin for stimulated groups was discovered (FIG. 6E). SEM imaging further confirmed the reduction in the porcine skin colonization by S. epidermidis, showing a substantial decrease in biofilm coverage (FIGS. 6F, 6G). Furthermore, it was examined whether BLAST could be applied to control S. epidermidis biofilm formation on other clinically relevant surfaces. Following an identical protocol, S. epidermidis was inoculated on the silicone surface utilized for catheter tubing and the surface of ultra-high molecular weight polyethylene utilized for prosthetic implants. Upon applying periodic electrical stimulation, significant reduction in the colony forming units was observed for stimulated groups (FIG. 26). This demonstration highlights a bioelectronic device exploiting selective excitability of the opportunistic pathogen, enabling drug-free control.

### Bioelectronic Localized Antimicrobial Stimulation Therapy (BLAST)

In some embodiments, a method of electrically stimulating a resident bacteria on an epidermis of an animal includes Bioelectronic Localized Antimicrobial Stimulation Therapy (BLAST). The method includes applying an acidic hydrogel to a region of the epidermis of the animal containing the resident bacteria, covering the region with a bioelectronic device, and administering an electrical stimulus through the bioelectronic device to the region such that the electrical stimulus exerts an antimicrobial effect on the resident bacteria.

In such embodiments, exciting the resident bacteria includes reducing a pathogenicity of the resident bacteria. Further, in some embodiments, reducing the pathogenicity of the resident bacteria includes eliciting a hyperpolarization response in the resident bacteria via the electrical stimulus such that eliciting the hyperpolarization response in the resident bacteria comprises reducing colonization capability of the resident bacteria. In other embodiments, reducing the pathogenicity of the resident bacteria comprises causing cytoplasmic acidification and reversible changes in a membrane potential of the resident bacteria such that causing cytoplasmic acidification and reversible changes in the membrane potential of the resident bacteria comprises suppressing growth and biofilm formation in the resident bacteria.

In some embodiments, administering the electrical stimulus through the bioelectronic device to the region comprises delivering alternating current at specified frequencies and voltages to the bioelectronic device via a potentiostat. For example, in some embodiments, a voltage of 1.5V_{ac} is applied to the bioelectronic device to elicit a hyperpolarization response in the resident bacteria.

In some embodiments, the bioelectronic device comprises a wearable electroceutical device, such as the wearable electroceutical device 100, shown in FIGS. 6A-6B. In some embodiments, the wearable electroceutical device includes a first layer being a wearable film dressing and a second layer applied to an interfacing side of the wearable film dressing. In such embodiments, the second layer includes a flexible polyimide substrate and an interdigitated electrode array integrated onto the flexible polyimide substrate. The second layer is configured to be applied to the region via the first layer.

In some embodiments, administering the electrical stimulus comprises delivering alternating current at specified frequencies and voltages to the second layer of the wearable electroceutical device.

In some embodiments, the resident bacteria comprise at least one of: Staphylococcus epidermidis, Staphylococcus capitis, and Staphylococcus saprophyticus. Further, in some embodiments, the animal is a mammal, such as a human.

### Apparatus-Wearable Electroceutical Device

In some embodiments, a wearable electroceutical device 100, as is shown in FIGS. 6A-6B, is used to electrically stimulate a resident bacteria 104 on an epidermis 102 of an animal includes Bioelectronic Localized Antimicrobial Stimulation Therapy (BLAST). In such embodiments, the wearable electroceutical device 100 includes a first layer 106 being a wearable film dressing, such as Tegaderm. Further, in such embodiments, the wearable electroceutical device 100 also includes a second layer 108 applied to an interfacing side of the wearable film dressing of the first layer 106. The second layer 108 includes a flexible polyimide substrate 110 and an interdigitated electrode array 112 integrated onto the flexible polyimide substrate 110. The second layer 108 is configured to be applied to a mammalian epidermis 102 via the first layer 106, and an acidic hydrogel interlayer 114 is configured to be disposed between the second layer 108 and the mammalian epidermis 102.

In some embodiments, the interdigitated electrode array 112 comprises gold electrodes. Further, in some embodiments, the interdigitated electrode array 112 enables electric potential localization between adjacent fingers of the electrodes in the interdigitated electrode array 112.

In some embodiments, the acidic hydrogel interlayer 114 comprises pH 5-adjusted tragacanth gum. In some embodiments, the acidic hydrogel interlayer 114 sensitizes the resident bacteria 104 on the mammalian epidermis 102 to electrical excitation. Further, in some embodiments, the resident bacteria 104 comprises at least one of: Staphylococcus epidermidis, Staphylococcus capitis, and Staphylococcus saprophyticus.

In some embodiments, a potentiostat is configured to deliver alternating current at specified frequencies and voltages to the wearable electroceutical device 100. For example, in some embodiments, a voltage of 1.5V_{ac} is applied to the wearable electroceutical device 100 to elicit a hyperpolarization response in the resident bacteria 104.

### Discussion

A microelectronic device was engineered to investigate the electrical excitability of the skin-residing opportunistic pathogen S. epidermidis. It was discovered that S. epidermidis can be excited by exogenous electrical stimuli, resulting in cytoplasmic acidification and reversible changes in membrane potential. The presence of a large transmembrane proton gradient near the epidermal pH conferred selective excitability to S. epidermidis and other skin-residing opportunistic pathogens. The bioelectrical stimulation programmably suppressed growth and biofilm formation in S. epidermidis. Finally, a drug-free electroceutical device was developed that exploits the selective excitability of S. epidermidis, reducing its colonization on a porcine skin model through BLAST treatment.

It was demonstrated that matching the acidic pH of the skin confers electrical excitability to opportunistic pathogens previously not known to be excitable. This “selective excitability” arises within a narrow pH range of 5-5.5, a hostile condition that deviates from the optimum growth pH for the organism. Given that bacteria have evolved mechanisms to maintain their H⁺ gradient under varying environments, each species may possess a different regime for electrical excitation. While B. subtilis and E. coli are the only two non-electroactive bacteria reported to be electrically excitable, selective excitability could be a key to uncover hidden excitatory responses in other microorganisms. Exploring the excitability of functional microbes may facilitate electrical control of bacterial physiology for diverse applications.

The finding of S. epidermidis' selective excitability was utilized to develop a device for controlling bacterial physiology and pathology. Through a low-voltage AC stimulation that is safe and imperceptible to humans, a non-lethal excitation response was elicited in S. epidermidis through reversible changes in membrane potential. This bioelectrical method was allowed to control growth, ATP, and biofilm formation of S. epidermidis at a benign voltage that was ineffective to the bacteria outside their selective pH. In the absence of the excitatory response, significantly higher voltage was required to achieve a similar level of biofilm suppression while risking device degradation. This method contrasts with conventional electrical stimulation typically used to either electrolytically kill bacteria through toxic chemicals or electroporate cells at dangerously high voltages. These results will promote further research into bioelectrical control of medically relevant bacteria, extending beyond lethal electroporation.

This bioelectronic treatment not only enables drug-free control of opportunistic pathogens but also demonstrates effectiveness and advantages over drug-based methods. Periodic electrical stimulation reduced biofilm formation by over 90%, with the reduction localized at the interdigitated electrodes. By programming the stimulation protocol, treatment can be transitioned from drug-enhanced to drug-free modes of biofilm suppression. In addition, growth, ATP levels, and antibiotic accumulation could be controlled stepwise by adjusting the number of stimulation cycles. The localized, programmable, and highly controllable therapy demonstrates the unique advantages of bioelectronic treatment over antibiotics-based methods. This approach is very easy to customize for individual patients and their treatment needs.

While it was demonstrated that an electroceutical device can be created that exploits the excitability of opportunistic pathogens, much more work is needed to apply the device in practical settings. Molecular-level studies are needed to identify which ion channels could be involved in mediating electrical responses in bacteria. Improved fundamental understanding of ionic responses could also enable an engineering approach to amplify the electrical response. Also, it should be noted that AC stimulation may be less convenient for wearable device applications due to the complexity of design and higher power consumption. To improve practicality, developing power-efficient methods and optimized circuit designs is essential. A better molecular understanding of bacterial stimulation mechanisms—including the role of ion channels and the contributions of capacitive and faradaic components—may enable the exploration of alternative signal types for stimulation.

Lastly, exploring the environmental factors that grant electrical responsivity to bacteria may offer valuable insights into the evolutionary interplay between microorganisms and their host environments. For example, the stable proton gradient at pH 5 suggests that S. epidermidis can endure unfavorable epidermal pH levels to maintain the driving force for ATP synthesis, surviving as a part of the commensal skin flora. From the host's perspective, evolution has maintained acidic skin pH, which is crucial for enzymatic regulation, barrier integrity, and microbial defense. However, the impact of acidic epidermal pH on conferring electrical excitability to bacterial inhabitants remains largely unexplored. The presence of natural electrical phenomena on the skin, such as trans-epidermal electric potential and electrostatic discharge, prompts intriguing questions regarding their potential influence on the electrophysiology of commensal microbes. Investigating this interaction warrants further scientific exploration.

### V. Materials and Methods

### Strains and Growth Conditions

Staphylococcus epidermidis (strain NIHLM087, provided by Dr. Julia Segre at NIH, NCBI taxonomy ID: NCBI: txid979201) was cultured overnight in tryptic soy broth (TSB) using a shaker incubator (37° C., 200 rpm). A 1 mL sample of the overnight liquid culture (OD₆₀₀˜3.00) was pelleted and resuspended in an equal volume of pH-adjusted TSB buffer. The resuspended bacterial solution was then added to an aerated culture tube and incubated in the shaker incubator (37° C., 200 rpm) for 3 hours.

For imaging membrane potential or antibiotics accumulation, 10 μM of Thioflavin T (ThT, Acros Organics), 200 nM of Trimethylrhodamine (TMRM) or 2 μg/mL of Gentamicin Texas Red (GTTR, AAT Bioquest) was added after 2 hours of incubation. After 3 hours, 1 μL of cells were inoculated onto pH-adjusted TSB agarose pads containing the same concentration of ThT or GTTR. To abolish the transmembrane pH gradient, the agarose pad and liquid culture were supplemented with 150 μM of CCCP. The agarose pad was prepared by melting ultrapure agarose (Invitrogen), which was then solidified on a 22 mm×22 mm cover glass and cut to an 8 mm×8 mm size. The inoculated agarose pad was placed on the interdigitated gold electrode device for imaging and stimulation.

For imaging of intracellular pH, 1 mL of the overnight cultured S. epidermidis was resuspended in 1 mL of pH 7.4 TSB. Then, 2 μL of pHrodo™ Green AM Ester and 20 μL of PowerLoad™ concentrate (Invitrogen) were added to the suspension and left at room temperature for 30 minutes. After this incubation, the cells loaded with pHrodo were washed twice with pH 7.4 TSB and resuspended in 1 mL of the respective pH-adjusted TSB buffers. The bacteria were incubated for 3 hours (37° C., 200 rpm) in an aerated culture tube and inoculated on a TSB agarose pad without any added dyes. The inoculated agarose pad was placed on the interdigitated gold electrode device for imaging and stimulation. An identical procedure was used for the culturing and stimulation of Staphylococcus capitis (ATCC #35661), Staphylococcus saprophyticus (ATCC #15305), and Escherichia coli (MG1655).

### Device Fabrication and Characterization

To fabricate the interdigitated stimulation device for assessment of bacterial excitability, #1.5 thickness microscope cover glass (Brain Research Lab, #4860-1.5D) was sonicated in acetone and isopropyl alcohol for 15 minutes. After blow drying with N₂, the cover glass was subjected to hexamethyldisilazane (HMDS) vapor priming. A SiO₂ wafer (NOVA Electronic Materials) of identical dimensions (60 mm×48 mm) was spin-coated with AZ nL of 2070 (2500 rpm, 45 s). Following HMDS treatment, the cover glass substrate was placed on top of the photoresist-coated SiO₂ wafer and baked at 110° C. for 3 minutes. The cover glass was bound to the wafer to reduce thermal expansion during subsequent fabrication processes. Next, the substrate was spin-coated with AZ nLOF 2020 and soft-baked at 110° C. for 2 minutes. The substrate was exposed to a 375 nm laser with a Heidelberg MLA150 Direct Write Lithographer. After a post-exposure bake at 110° C. for 2 minutes, the pattern was developed in AZ 300 MIF for 50 seconds. After washing in DI water, 10 nm of Ti and 100 nm of gold were deposited using an Evo V_{ac} Electron Beam Evaporator (Angstrom). The photoresist and bonding to the SiO₂ wafer were lifted off in PG remover at 80° C. overnight, releasing the cover glass substrate with interdigitated gold electrodes.

Acrylic plastic (Alfa Acsar) was cut with a VLS 4.60 laser cutter to make a 2×2 well, which was bound to the cover glass substrate using Kwik-Sil™ (World Precision Instruments). The device was wired using copper wire (Remington) and PELCOR conductive silver paint (Ted Pella), which was insulated with epoxy (JB Weld). For the fabrication of the electroceutical skin patch, an identical procedure was used, replacing the cover glass substrate with polyimide (PI) film up to the lift-off stage using PG remover. The released PI film with interdigitated gold electrodes was wired and attached to Tegaderm (3M). The dimensions of the interdigitated electrode was checked using LEXT OLS5100 confocal microscope (Olympus) and Merlin SEM (ZEISS) and processed with the manufacturer's software.

### Finite Element Simulations

Finite element analysis of the electric field distribution was conducted using COMSOL Multiphysics software. COMSOL simulation of the electric potential distribution occurs as described below.

The electric potential distribution across the zy axis of the interdigitated microelectrode was analyzed using finite element analysis in the AC/DC module of COMSOL Multiphysics. Poisson's equation, −∇·(ε0εr∇V)=ρ, governs the electric scalar potential, V, where ε0 is the permittivity of free space, εr is the relative permittivity, and ρ is the space charge density. The electric field, E, is derived from the gradient of V as E=−∇V, and the electric displacement, D, is defined as D=ε0εrE. The model features interdigitated electrodes with a width and gap of 40 μm, matching the device specifications. Two 2D simulations were performed across the ZY and XY planes to obtain the electric field profiles in the top and bottom images respectively. The simulations were performed with 0V and +1.5V constraints for the interdigitated electrodes, and a zero-charge boundary condition for the remaining boundaries. The surrounding medium was modeled as a dielectric with a relative permittivity of 80, which approximates the values for culture media. For the simulation in the XY plane, the metal region was omitted from the simulation, and boundary conditions were set for the metal-media interface.

Electrical Stimulation of S. epidermidis

Electric stimulation was applied using an SP200 Potentiostat (BioLogic). One stimulation cycle consisted of 75 mVpp/μm, AC, 0.1 kHz for 10 seconds. For enhancing the inhibitory effect of antibiotics, S. epidermidis was preconditioned with five cycles of stimulation (1-minute interval) and then incubated with a sublethal concentration (30 μg/mL) of gentamicin for 18 hours at 37° C. For drug-free biofilm suppression, stimulation cycles were continuously applied at 10-minute intervals for 18 hours at 37° C.

### Fluorescence Microscopy

Short-term timelapse images with an imaging duration below 10 minutes were acquired using a Stellaris 8 WLL confocal microscope (Leica Microsystems) with a 63× objective (63×/1.4 UV oil 0.14 mm WD, 506350). ThT fluorescence was detected with an excitation laser of 458 nm and an emission detection window of 490 nm to 545 nm using the HyD X2 detector. pHrodo fluorescence was detected with an excitation laser of 500 nm and an emission detection window of 530 nm to 600 nm using the HyD X2 detector. GTTR fluorescence was detected with an excitation laser of 595 nm and an emission detection window of 630 nm to 750 nm using the HyD S3 detector. Imaging of bacterial viability was done using the LIVE/DEAD BacLight Bacterial Viability Kit (Molecular Probes) following the provider's guidelines. For overnight timelapse experiments, phase contrast images of S. epidermidis were captured using an Axio Observer 7 microscope (Zeiss) with a 100× or 63× objective (1.4 oil immersion) in an incubator box maintained at 37° C. Images were background subtracted and adjusted for brightness and contrast using ImageJ.

Intracellular pH Assay with BCECF-AM

1 mL of the overnight cultured S. epidermidis was pelleted and resuspended in an equal volume of pH-adjusted TSB buffers. The resuspended bacterial solution was added to an aerated culture tube and incubated in a shaker incubator (37° C., 200 rpm) for 3 hours. After the incubation, 25 μM of BCECF-AM (Invitrogen) was added to the culture tubes and incubated at 30° C. for 30 minutes. BCECF-AM fluorescence was measured using a Synergy Neo HTS Plate Reader according to the manufacturer's instructions. The Intracellular pH Calibration Buffer Kit (P35379) was used to calibrate BCECF-AM fluorescence with intracellular pH, allowing for the quantification of transmembrane pH gradient, ΔpH. Transmembrane proton gradient, ΔH⁺, was measured by taking negative anti-log of pH to find proton concentration [H⁺], to calculate ΔH⁺=[H⁺]ₑₓₜₑᵣₙₐₗ−[H⁺]ᵢₙₜₑᵣₙₐₗ. For the kinetics experiment using BCECF-AM, fluorescence measurements were taken every minute in the Synergy Neo HTS Plate Reader. After 5 minutes, the plate was taken out and 150 μM CCCP or 3 μM Nigericin (Invitrogen) was added to the wells. Upon addition, changes in BCECF-AM fluorescence were continuously monitored for 30 minutes.

### Measurement of ATP and Growth

Upon applying the desired cycles of electrical stimulation, the agarose pad and electrode surface were flushed with TSB medium to collect S. epidermidis. Using a Synergy Neo HTS Plate Reader, the optical density of the stimulated and non-stimulated cells was adjusted to OD₆₀₀=0.2. ATP levels in stimulated cells were measured using the BacTiter-Glo™ Microbial Cell Viability Assay and normalized to those of non-stimulated controls. For the measurement of growth, 200 μL of the OD₆₀₀=0.2 samples were added to a 96-well plate, and optical density was monitored overnight using a Tecan Infinite 200 microplate reader at 37° C. under orbital shaking.

### Real-Time Quantitative Reverse Transcription PCR

Upon applying ten cycles of electric stimulation at 60-second intervals, the agarose pad and electrode were flushed with TSB medium to collect stimulated S. epidermidis. Using a Synergy Neo HTS Plate Reader, the optical density of the stimulated and non-stimulated cells was adjusted to OD₆₀₀=0.03. Then, 200 μL of the cells were added to a 96-well plate and incubated for 8 hours at 37° C. with orbital shaking in a Tecan Infinite 200 microplate reader, until they reached the midexponential growth phase. Subsequently, RNA was extracted using the Qiagen RNeasy Mini Kit (Cat. No. 74104).

Quantitative RT-PCR was performed using the One-Step TB Green® PrimeScript™ RT-PCR Kit II (Takara, Cat. No. RR086A) and a QuantStudio Pro 6 PCR system (Applied Biosystems). ΔCt values were calculated relative to the guanylate kinase (gmk) housekeeping gene, and fold changes were calculated relative to the non-stimulated control samples. The virulence gene sequence of S. epidermidis was obtained from the NCBI database, and primers were designed and evaluated using Primer3 software.

### Primer Sequences:

icaC-u:
(SEQ ID NO: 1)
TCACTACCGGAAACAGCGAT,
icaC-d:
(SEQ ID NO: 2)
TTCTTAGGTGGTTACATTGGCT
icaB-u:
(SEQ ID NO: 3)
ACTTTGCGTCGTGTGCTTTA,
icaB-d:
(SEQ ID NO: 4)
GCACTCGCGTTAAACTATCAC
Psm-u:
(SEQ ID NO: 5)
CGCTAACGCCACTTTCTACG,
Psm-d:
(SEQ ID NO: 6)
GCAGCACAAGATCAAGATTGG
gmk-u:
(SEQ ID NO: 7)
CCTTCAGGTGTTGGAAAGG,
gmk-d:
(SEQ ID NO: 8)
CACCTTCACGCATATGACGC
Clp-u:
(SEQ ID NO: 9)
ACATAAACTCGTCGGGTGGT,
Clp-d:
(SEQ ID NO: 10)
AGCGATCACACTTGCGATTG
MrSA-u:
(SEQ ID NO: 11)
TCTTGATGGTATGCTTCCGC,
MrSA-d:
(SEQ ID NO: 12)
TCCTTTGGATGATGGTGGTC
SarA-u:
(SEQ ID NO: 13)
TGCTTCTGTGATACGGTTGT,
SarA-d:
(SEQ ID NO: 14)
TGAACACGATGAGAGAACTGTT
SdrG-u:
(SEQ ID NO: 15)
CAACAAGTCAGCCGTCTAGC,
SdrG-d:
(SEQ ID NO: 16)
CTTGTTCCGCCGCTAATTGA
Acc-u:
(SEQ ID NO: 17)
TGTTTCTTAGTTGCTGCAGGT,
Acc-d:
(SEQ ID NO: 18)
TTAGGAGACATGCACACGGT

### Electrical Stimulation & Characterization of Porcine Skin

Porcine skin (Fisher Scientific, NC1275387) was soaked in TSB buffer overnight before the experiment. 3 μL of S. epidermidis was inoculated onto the porcine skin surface. pH 5-adjusted tragacanth gum (3% w/v, Sigma-Aldrich) was applied to the electrode surface (8 μL/cm²). Finally, the electrode was placed on the inoculated porcine skin, and a periodic electrical stimulation was applied, consisting of stimulation every 10 minutes for 18 hours at 37° C. To quantify colony-forming units on the porcine skin, the skin and electrode were flushed with pH 7.4 TSB to collect attached bacteria. The TSB containing the collected S. epidermidis was incubated for 6 hours in an aerated bacterial culture tube before being inoculated on a TSB agar plate for colony counting.

To determine the pH of porcine skin before and after applying tragacanth gum, a 1 cm² piece of skin was soaked in PBS at pH 7.4 overnight. After soaking, the skin was gently shaken to remove excess PBS, and MQuant® pH-indicator strips (range: pH 5-10) were placed on the skin's surface. Then, 8 L/cm² of pH 5 tragacanth gum was spread on the skin, and the pH was allowed to equilibrate for 10 minutes. Excess gum was gently dried off with a towel, and another pH-indicator strip was placed on the skin. The strip's color was analyzed by photographing it, converting the image to HSB stack in ImageJ, and quantifying the hue, saturation, and brightness. The quantified color was then compared to control strips applied to pH 5 PBS and pH 5-tragacanth gum.

For scanning electron microscopy (SEM) characterization, the porcine skin was fixed with 3% glutaraldehyde for 16 hours at 4° C. The fixed porcine skin was subjected to increasing concentrations of acetone and IPA solvent series and dried with a Leica EM CPD300 Critical Point Drier following the manufacturer's protocol on preparing bacterial samples. The dried samples were sputtered with 8 nm of Pt/Pd using a Cressington Sputter Coater 208 and imaged with a Merlin FE SEM (Zeiss).

### Statistical Analyses

OriginPro 2024 was employed for all statistical analyses. Unless stated otherwise, the error bars represent a standard deviation. For biological data analysis, multiple t-tests were performed. A significance threshold of P<0.05 was used to determine statistical significance.

### REFERENCES

1. Li, P., Kim, S., and Tian, B. (2024). Beyond 25 years of biomedical Innovation in nano-bioelectronics. Device 2, 100401. doi.org/10.1016/j.device.2024.100401.
2. Li, P., Zhang, J., Hayashi, H., Yue, J., Li, W., Yang, C., Sun, C., Shi, J., Huberman-Shlaes, J., Hibino, N., et al. (2024). Monolithic silicon for high spatiotemporal translational photostimulation. Nature 626, 990-998. doi.org/10.1038/s41586-024-07016-9.
3. Prominski, A., Shi, J., Li, P., Yue, J., Lin, Y., Park, J., Tian, B., and Rotenberg, M. Y. (2022). Porosity-based heterojunctions enable leadless optoelectronic modulation of tissues. Nat. Mater. 21, 647-655. doi.org/10.1038/s41563-022-01249-7.
4. Nirenberg, S., and Pandarinath, C. (2012). Retinal prosthetic strategy with the capacity to restore normal vision. Proc. Natl. Acad. Sci. U.S.A 109, 15012-15017. doi.org/10.1073/pnas. 1207035109.
5. Guyot, M., Simon, T., Ceppo, F., Panzolini, C., Guyon, A., Lavergne, J., Murris, E., Daoudlarian, D., Brusini, R., Zarif, H., et al. (2019). Pancreatic nerve electrostimulation inhibits recent-onset autoimmune diabetes. Nat. Biotechnol. 37, 1446-1451. doi.org/10.1038/s41587-019-0295-8.
6. Balasubramanian, S., Weston, D. A., Levin, M., and Davidian, D. C. C. (2024). Charging Ahead: Examining the Future Therapeutic Potential of Electroceuticals. Adv. Ther. 7, 2300344. doi.org/10.1002/adtp.202300344.
7. Balasubramanian, S., Weston, D. A., Levin, M., and Davidian, D. C. C. (2024). Electroceuticals: emerging applications beyond the nervous system and excitable tissues. Trends Pharmacol. Sci. 45, 391-394. doi.org/10.1016/j.tips.2024.03.001.
8. Prindle, A., Liu, J., Asally, M., Ly, S., Garcia-Ojalvo, J., and Süel, G. M. (2015). Ion channels enable electrical communication in bacterial communities. Nature 527, 59-63. doi.org/10.1038/nature15709.
9. Erin Chen, Y., Fischbach, M. A., and Belkaid, Y. (2018). Skin microbiota-host interactions. Nature 553, 427-436. doi.org/10.1038/nature25177.
10. Shi, J., Kim, S., Li, P., Dong, F., Yang, C., Nam, B., Han, C., Eig, E., Shi, L. L., Niu, S., et al. (2024). Active biointegrated living electronics for managing inflammation. Science (80-.). 384, 1023-1030. doi.org/10.1126/science.adl1102.
11. Severn, M. M., and Horswill, A. R. (2023). Staphylococcus epidermidis and its dual lifestyle in skin health and infection. Nat. Rev. Microbiol. 21, 97-111. doi.org/10.1038/s41579-022-00780-3.
12. Otto, M. (2009). Staphylococcus epidermidis—The “accidental” pathogen. Nat. Rev. Microbiol. 7, 555-567. doi.org/10.1038/nrmicro2182.
13. Dong, Y., Speer, C. P., and Glaser, K. (2018). Beyond sepsis: Staphylococcus epidermidis is an underestimated but significant contributor to neonatal morbidity. Virulence 9, 621-633. doi.org/10.1080/21505594.2017.1419117.
14. Landemaine, L., Da Costa, G., Fissier, E., Francis, C., Morand, S., Verbeke, J., Michel, M. L., Briandet, R., Sokol, H., Gueniche, A., et al. (2023). Staphylococcus epidermidis isolates from atopic or healthy skin have opposite effect on skin cells: potential implication of the AHR pathway modulation. Front. Immunol. 14, 1-19. doi.org/10.3389/fimmu.2023.1098160.
15. Hon, K. L., Tsang, Y. C. K., Pong, N. H., Leung, T. F., and Ip, M. (2016). Exploring Staphylococcus epidermidis in atopic eczema: friend or foe? Clin. Exp. Dermatol. 41, 659-663. doi.org/10.1111/ced.12866.
16. Clavaud, C., Jourdain, R., Bar-Hen, A., Tichit, M., Bouchier, C., Pouradier, F., El Rawadi, C., Guillot, J., Ménard-Szczebara, F., Breton, L., et al. (2013). Dandruff Is Associated with Disequilibrium in the Proportion of the Major Bacterial and Fungal Populations Colonizing the Scalp. PLOS One 8. doi.org/10.1371/journal.pone.0058203.
17. Cunha, B. A. (2001). antibiotic side effects. Med. Clin. North Am. 85, 149-185. doi.org/10.1016/s0025-7125 (05) 70309-6.
18. Weidinger, S., Beck, L. A., Bieber, T., Kabashima, K., and Irvine, A. D. (2018). Atopic dermatitis. Nat. Rev. Dis. Prim. 4. doi.org/10.1038/s41572-018-0001-z.
19. Larsson, D. G. J., and Flach, C. F. (2022). Antibiotic resistance in the environment. Nat. Rev. Microbiol. 20, 257-269. doi.org/10.1038/s41579-021-00649-x.
20. Darby, E. M., Trampari, E., Siasat, P., Gaya, M. S., Alav, I., Webber, M. A., and Blair, J. M. A. (2023). Molecular mechanisms of antibiotic resistance revisited. Nat. Rev. Microbiol. 21, 280-295. doi.org/10.1038/s41579-022-00820-y.
21. Lee, J. Y. H., Monk, I. R., Gonçalves da Silva, A., Seemann, T., Chua, K. Y. L., Kearns, A., Hill, R., Woodford, N., Bartels, M. D., Strommenger, B., et al. (2018). Global spread of three multidrug-resistant lineages of Staphylococcus epidermidis. Nat. Microbiol. 3, 1175-1185. doi.org/10.1038/s41564-018-0230-7.
22. Koo, J., MacEwan, M. R., Kang, S. K., Won, S. M., Stephen, M., Gamble, P., Xie, Z., Yan, Y., Chen, Y. Y., Shin, J., et al. (2018). Wireless bioresorbable electronic system enables sustained nonpharmacological neuroregenerative therapy. Nat. Med. 24, 1830-1836. doi.org/10.1038/s41591-018-0196-2.
23. Pavlov, V. A., and Tracey, K. J. (2022). Bioelectronic medicine: Preclinical insights and clinical advances. Neuron 110, 3627-3644. doi.org/10.1016/j.neuron.2022.09.003.
24. Moritz, C., Field-Fote, E. C., Tefertiller, C., van Nes, I., Trumbower, R., Kalsi-Ryan, S., Purcell, M., Janssen, T. W. J., Krassioukov, A., Morse, L. R., et al. (2024). Noninvasive spinal cord electrical stimulation for arm and hand function in chronic tetraplegia: a safety and efficacy trial. Nat. Med. 30, 1276-1283. doi.org/10.1038/s41591-024-02940-9.
25. Sultana, S. T., Babauta, J. T., and Beyenal, H. (2015). Electrochemical biofilm control: A review. Biofouling 31, 745-758. doi.org/10.1080/08927014.2015.1105222.
26. Czerwińska-Główka, D., and Krukiewicz, K. (2020). A journey in the complex interactions between electrochemistry and bacteriology: From electroactivity to electromodulation of bacterial biofilms. Bioelectrochemistry 131, 107401. doi.org/10.1016/j.bioelechem.2019.107401.
27. Kim, S., Eig, E., and Tian, B. (2024). The convergence of bioelectronics and engineered living materials. Cell Reports Phys. Sci. 5, 102149. doi.org/10.1016/j.xcrp.2024.102149.
28. Li, P., Kim, S., and Tian, B. (2022). Nanoenabled Trainable Systems: From Biointerfaces to Biomimetics. ACS Nano 16, 19651-19664. doi.org/10.1021/acsnano.2c08042.
29. Benarroch, J. M., and Asally, M. (2020). The Microbiologist's Guide to Membrane Potential Dynamics. Trends Microbiol. 28, 304-314. doi.org/10.1016/j.tim.2019.12.008.
30. Jones, J. M., and Larkin, J. W. (2021). Toward Bacterial Bioelectric Signal Transduction. Bioelectricity 3, 116-119. doi.org/10.1089/bioe.2021.0013.
31. Stratford, J. P., Edwards, C. L. A., Ghanshyam, M. J., Malyshev, D., Delise, M. A., Hayashi, Y., and Asally, M. (2019). Electrically induced bacterial membrane-potential dynamics correspond to cellular proliferation capacity. Proc. Natl. Acad. Sci. U.S.A 116, 9552-9557. doi.org/10.1073/pnas. 1901788116.
32. Sender, R., Fuchs, S., and Milo, R. (2016). Are We Really Vastly Outnumbered? Revisiting the Ratio of Bacterial to Host Cells in Humans. Cell 164, 337-340. doi.org/10.1016/j.cell.2016.01.013.
33. Larkin, J. W., Zhai, X., Kikuchi, K., Redford, S. E., Prindle, A., Liu, J., Greenfield, S., Walczak, A. M., Garcia-Ojalvo, J., Mugler, A., et al. (2018). Signal Percolation within a Bacterial Community. Cell Syst. 7, 137-145.e3. doi.org/10.1016/j.cels.2018.06.005.
34. Yang, C. Y., Bialecka-Fornal, M., Weatherwax, C., Larkin, J. W., Prindle, A., Liu, J., Garcia-Ojalvo, J., and Süel, G. M. (2020). Encoding Membrane-Potential-Based Memory within a Microbial Community. Cell Syst. 10, 417-423.e3. doi.org/10.1016/j.cels.2020.04.002.
35. Comerci, C. J., Gillman, A. L., Galera-Laporta, L., Gutierrez, E., Groisman, A., Larkin, J. W., Garcia-Ojalvo, J., and Süel, G. M. (2022). Localized electrical stimulation triggers cell-type-specific proliferation in biofilms. Cell Syst. 13, 488-498.e4. doi.org/10.1016/j.cels.2022.04.001.
36. Fang, Y., Prominski, A., Rotenberg, M. Y., Meng, L., Acarón Ledesma, H., Lv, Y., Yue, J., Schaumann, E., Jeong, J., Yamamoto, N., et al. (2021). Micelle-enabled selfassembly of porous and monolithic carbon membranes for bioelectronic interfaces. Nat. Nanotechnol. 16, 206-213. doi.org/10.1038/s41565-020-00805-z.
37. Ortega, M. A., Fernández-Garibay, X., Castaño, A. G., De Chiara, F., Hernández-Albors, A., Balaguer-Trias, J., and Ramón-Azcón, J. (2019). Muscle-on-a-chip with an on-site multiplexed biosensing system for: In situ monitoring of secreted IL-6 and TNF-α. Lab Chip 19, 2568-2580. doi.org/10.1039/c91c00285e.
38. Liu, N., and Gao, Y. (2017). Recent Progress in Micro-Supercapacitors with In-Plane Interdigital Electrode Architecture. Small 13, 1-10.doi.org/10.1002/smll.201701989.
39. Chiera, S., Bosco, F., Mollea, C., Piscitello, A., Sethi, R., Nollo, G., Caola, I., and Tessarolo, F. (2023). Staphylococcus epidermidis is a safer surrogate of Staphylococcus aureus in testing bacterial filtration efficiency of face masks. Sci. Rep. 13, 1-10. doi.org/10.1038/s41598-023-49005-4.
40. Lambers, H., Piessens, S., Bloem, A., Pronk, H., and Finkel, P. (2006). Natural skin surface pH is on average below 5, which is beneficial for its resident flora. Int. J. Cosmet. Sci. 28, 359-370. doi.org/10.1111/j.1467-2494.2006.00344.x.
41. O'Goshi, K. ichiro, Okada, M., Iguchi, M., and Tagami, H. (2002). The predilection sites for chronic atopic dermatitis do not show any special functional uniqueness of the stratum corneum: Measurements of the water barrier function, hydration state, skin surface lipids, corneocyte size and pH in patients and normal indi. Exog. Dermatology 1, 195-202. doi.org/10.1159/000066233.
42. Prakash, C., Bhargave, P., Tiwari, S., Majumdar, B., and Bhargava, R. K. (2017). Skin Surface pH in Acne Vulgaris: Insights from an Observational Study and Review of the Literature. J. Clin. Aesthet. Dermatol. 10, 33-39.
43. Sim, P., Strudwick, X. L., Song, Y. M., Cowin, A. J., and Garg, S. (2022). Influence of Acidic pH on Wound Healing In Vivo: A Novel Perspective for Wound Treatment. Int. J. Mol. Sci. 23, 13655. doi.org/10.3390/ijms232113655.
44. Proksch, E. (2018). pH in nature, humans and skin. J. Dermatol. 45, 1044-1052. doi.org/10.1111/1346-8138.14489.
45. Lukić, M., Pantelić, I., and Savić, S. D. (2021). Towards optimal pH of the skin and topical formulations: From the current state of the art to tailored products. Cosmetics 8, 69. doi.org/10.3390/cosmetics8030069.
46. Wang, T., Chen, H., Yu, C., and Xie, X. (2019). Rapid determination of the electroporation threshold for bacteria inactivation using a lab-on-a-chip platform. Environ. Int. 132, 105040. doi.org/10.1016/j.envint.2019.105040.
47. Dorr, D. (2009). Determining Voltage Levels of Concern for Human and Animal Response to AC Current. IEEE, 1-6. doi.org/10.1109/PES.2009.5275848.
48. Krulwich, T. A., Sachs, G., and Padan, E. (2011). Molecular aspects of bacterial pH sensing and homeostasis. Nat. Rev. Microbiol. 9, 330-343. doi.org/10.1038/nrmicro2549.
49. Padan, E., Zilberstein, D., and Schuldiner, S. (1981). pH homesstasis in bacteria. Biochim. Biophys. Acta 650, 151-166. doi.org/10.1016/0304-4157 (81) 90004-6.
50. Chong, C. E., Bengtsson, R. J., and Horsburgh, M. J. (2022). Comparative genomics of Staphylococcus capitis reveals species determinants. Front. Microbiol. 13, 1005949. doi.org/10.3389/fmich.2022.1005949.
51. Lawal, O. U., Barata, M., Fraqueza, M. J., Worning, P., Bartels, M. D., Goncalves, L., Paixão, P., Goncalves, E., Toscano, C., Empel, J., et al. (2021). Staphylococcus saprophyticus From Clinical and Environmental Origins Have Distinct Biofilm Composition. Front. Microbiol. 12, 1-13. doi.org/10.3389/fmicb.2021.663768.
52. Lagadic-Gossmann, D., Huc, L., and Lecureur, V. (2004). Alterations of intracellular pH homeostasis in apoptosis: Origins and roles. Cell Death Differ. 11, 953-961. doi.org/10.1038/sj.cdd.4401466.
53. Lee, D. yeon D., Galera-Laporta, L., Bialecka-Fornal, M., Moon, E. C., Shen, Z., Briggs, S. P., Garcia-Ojalvo, J., and Süel, G. M. (2019). Magnesium Flux Modulates Ribosomes to Increase Bacterial Survival. Cell 177, 352-360.e13. doi.org/10.1016/j.cell.2019.01.042.
54. Tormo, M. A., Marti, M., Valle, J., Manna, A. C., Cheung, A. L., Lasa, I., and Penades, J. R. (2005). SarA is an essential positive regulator of Staphylococcus epidermidis biofilm development. J. Bacteriol. 187, 2348-2356. doi.org/10.1128/JB.187.7.2348-2356.2005.
55. Wang, C., Li, M., Dong, D., Wang, J., Ren, J., Otto, M., and Gao, Q. (2007). Role of ClpP in biofilm formation and virulence of Staphylococcus epidermidis. Microbes Infect. 9, 1376-1383. doi.org/10.1016/j.micinf.2007.06.012.
56. Fey, P. D., and Olson, M. E. (2010). Current concepts in biofilm formation of Staphylococcus epidermidis. Future Microbiol. 5, 917-933. doi.org/10.2217/fmb.10.56.
57. Williams, M. R., Bagood, M. D., Enroth, T. J., Bunch, Z. L., Jiang, N., Liu, E., Almoughrabie, S., Khalil, S., Li, F., Brinton, S., et al. (2023). Staphylococcus epidermidis activates keratinocyte cytokine expression and promotes skin inflammation through the production of phenol-soluble modulins. Cell Rep. 42, 113024. doi.org/10.1016/j.celrep.2023.113024.
58. Gawande, P. V., Leung, K. P., and Madhyastha, S. (2014). Antibiofilm and antimicrobial efficacy of Dispersinb®-KSL-w peptide-based wound gel against chronic wound infection associated bacteria. Curr. Microbiol. 68, 635-641. doi.org/10.1007/s00284-014-0519-6.
59. Brackman, G., De Meyer, L., Nelis, H. J., and Coenye, T. (2013). Biofilm inhibitory and eradicating activity of wound care products against Staphylococcus aureus and Staphylococcus epidermidis biofilms in an in vitro chronic wound model. J. Appl. Microbiol. 114, 1833-1842. doi.org/10.1111/jam.12191.
60. Schierle, C. F., De La Garza, M., Mustoe, T. A., and Galiano, R. D. (2009). Staphylococcal biofilms impair wound healing by delaying reepithelialization in a murine cutaneous wound model. Wound Repair Regen. 17, 354-359. doi.org/10.1111/j.1524-475X.2009.00489.x.
61. Taghavizadeh Yazdi, M. E., Nazarnezhad, S., Mousavi, S. H., Sadegh Amiri, M., Darroudi, M., Baino, F., and Kargozar, S. (2021). Gum tragacanth (Gt): A versatile biocompatible material beyond borders. Molecules 26, 1510. doi.org/10.3390/molecules26061510.
62. Uhm, C., Jeong, H., Lee, S. H., Hwang, J. S., Lim, K. M., and Nam, K. T. (2023). Comparison of structural characteristics and molecular markers of rabbit skin, pig skin, and reconstructed human epidermis for an ex vivo human skin model. Toxicol. Res. 39, 477-484. doi.org/10.1007/s43188-023-00185-1.
63. Michels, M., and Bakker, E. P. (1985). Generation of a large, protonophore-sensitive proton motive force and pH difference in the acidophilic bacteria Thermoplasma acidophilum and Bacillus acidocaldarius. J. Bacteriol. 161, 231-237. doi.org/10.1128/jb.161.1.231-237.1985.
64. Mulkidjanian, A. Y., Dibrov, P., and Galperin, M. Y. (2008). The past and present of sodium energetics: May the sodium-motive force be with you. Biochim. Biophys. Acta 1777, 985-992. doi.org/10.1016/j.bbabio.2008.04.028.
65. Pirbadian, S., Chavez, M. S., and El-Naggar, M. Y. (2020). Spatiotemporal mapping of bacterial membrane potential responses to extracellular electron transfer. Proc. Natl. Acad. Sci. U.S.A 117, 20171-20179. doi.org/10.1073/PNAS.2000802117.
66. Liu, X., Gao, H., Ward, J. E., Liu, X., Yin, B., Fu, T., Chen, J., Lovley, D. R., and Yao, J. (2020). Power generation from ambient humidity using protein nanowires. Nature 578, 550-554. doi.org/10.1038/s41586-020-2010-9.
67. Shi, J., Lin, Y., Li, P., Mickel, P., Sun, C., Parekh, K., Ma, J., Kim, S., Ashwood, B., Meng, L., et al. (2024). Monolithic-to-focal evolving biointerfaces in tissue regeneration and bioelectronics. Nat. Chem. Eng. 1, 73-86. doi.org/10.1038/s44286-023-00008-y.
68. Li, Z., Misra, R. P., Li, Y., Yao, Y. C., Zhao, S., Zhang, Y., Chen, Y., Blankschtein, D., and Noy, A. (2023). Breakdown of the Nernst-Einstein relation in carbon nanotube porins. Nat. Nanotechnol. 18, 177-183. doi.org/10.1038/s41565-022-01276-0.
69. Tang, T. C., An, B., Huang, Y., Vasikaran, S., Wang, Y., Jiang, X., Lu, T. K., and Zhong, C. (2021). Materials design by synthetic biology. Nat. Rev. Mater. 6, 332-350. doi.org/10.1038/s41578-020-00265-w.
70. Long, Y., Wei, H., Li, J., Yao, G., Yu, B., Ni, D., Gibson, A. L., Lan, X., Jiang, Y., Cai, W., et al. (2018). Effective Wound Healing Enabled by Discrete Alternative Electric Fields from Wearable Nanogenerators. ACS Nano 12, 12533-12540. doi.org/10.1021/acsnano.8b07038.
71. Huang, Y., Yao, K., Zhang, Q., Huang, X., Chen, Z., Zhou, Y., and Yu, X. (2024). Bioelectronics for electrical stimulation: materials, devices and biomedical applications. Chem. Soc. Rev. 53, 8632-8712. doi.org/10.1039/d4cs00413b.
72. Saw, T. B., Gao, X., Li, M., He, J., Le, A. P., Marsh, S., Lin, K. hui, Ludwig, A., Prost, J., and Lim, C. T. (2022). Transepithelial potential difference governs epithelial homeostasis by electromechanics. Nat. Phys. 18, 1122-1128. doi.org/10.1038/s41567-022-01657-1.
73. Dawson, T. W., Stuchly, M. A., and Kavet, R. (2004). Evaluation of interactions of electric fields due to electrostatic discharge with human tissue. IEEE Trans. Biomed. Eng. 51, 2194-2198. doi.org/10.1109/TBME.2004.834293.
74. Vandecasteele, S. J., Peetermans, W. E., Merckx, R., and Van Eldere, J. (2001). Quantification of expression of Staphylococcus epidermidis housekeeping genes with Taqman quantitative PCR during in vitro growth and under different conditions. J. Bacteriol. 183, 7094-7101. doi.org/10.1128/JB.183.24.7094-7101.2001.
75. Stratford, J. P., Edwards, C. L. A., Ghanshyam, M. J., Malyshev, D., Delise, M. A., Hayashi, Y., and Asally, M. (2019). Electrically induced bacterial membrane-potential dynamics correspond to cellular proliferation capacity. PNAS 116, 9552-9557. doi.org/10.1073/pnas. 1901788116.
76. Krulwich, T. A., Sachs, G., and Padan, E. (2011). Molecular aspects of bacterial pH sensing and homeostasis. Nat. Rev. Microbiol. 9, 330-343. doi.org/10.1038/nrmicro2549.
77. Umeyama, H., Shigemi, Z., Baba, Y., Hara, N., Watanabe, T., and Fujimuro, M. (2023). Nigericin Induces Apoptosis in Primary Effusion Lymphoma Cells by Mitochondrial Membrane Hyperpolarization and β-Catenin Destabilization. Anticancer Res. 43, 2455-2465. doi.org/10.21873/anticanres. 16413.
78. Gao, G., Liu, F., Xu, Z., Wan, D., Han, Y., Kuang, Y., Wang, Q., and Zhi, Q. (2021). Evidence of nigericin as a potential therapeutic candidate for cancers: A review. Biomed. Pharmacother. 137, 111262. doi.org/10.1016/j.biopha.2021.111262.
79. Chen, M.-T., Jiang, C., Vernier, P. T., Wu, Y.-H., and Gundersen, M. A. (2009). Two dimensional nanosecond electric field mapping based on cell electropermeabilization. PMC Biophys. 2, 1-16. doi.org/10.1186/1757-5036-2-9.

1. A method of electrically stimulating a resident bacteria on an epidermis of an animal, the method comprising:
applying an acidic hydrogel to a region of the epidermis of the animal containing the resident bacteria;
covering the region with a bioelectronic device; and
administering an electrical stimulus through the bioelectronic device to the region, wherein the electrical stimulus exerts an antimicrobial effect on the resident bacteria.

2. The method of claim 1, wherein the acidic hydrogel comprises a natural and/or synthetic polymer hydrogel with a pH of about 5.

3. The method of claim 1, wherein the acidic hydrogel comprises pH 5-adjusted tragacanth gum.

4. The method of claim 1, wherein the bioelectronic device comprises a wearable electroceutical device, comprising:
a first layer, wherein the first layer is a wearable film dressing; and
a second layer applied to an interfacing side of the wearable film dressing, the second layer comprising:
a flexible polymer substrate, and
an electrode array integrated onto the flexible polymer substrate, wherein the second layer is configured to be applied to the region via the first layer.

5. The method of claim 1, wherein administering the electrical stimulus through the bioelectronic device to the region comprises delivering alternating current to the bioelectronic device via a potentiostat connected to the bioelectronic device.

6. The method of claim 5, wherein a voltage of 1.5V_{ac} is delivered by the potentiostat to elicit a hyperpolarization response in the resident bacteria.

7. The method of claim 1, wherein the antimicrobial effect comprises a reduction in pathogenicity of the resident bacteria.

8. The method of claim 7, wherein the reduction in pathogenicity of the resident bacteria comprises a reduction in growth of the resident bacteria.

9. The method of claim 8, wherein the reduction in growth of the resident bacteria comprises at least one of:
suppresses biofilm formation of the resident bacteria,
reduces expression of virulence genes responsible for surface adhesion (SdrG), polysaccharide intracellular adhesin (icaB, icaC), protease (Clp), antibiotic resistance (MrSA), and virulence regulation (SarA), and
reduces colonization capability of the resident bacteria.

10. The method of claim 1, wherein the resident bacteria comprises at least one of: Staphylococcus epidermidis, Staphylococcus capitis, and Staphylococcus saprophyticus.

11. The method of claim 1, wherein the animal is a mammal.

12. A wearable electroceutical device, comprising:
a first layer, wherein the first layer is a wearable film dressing; and
a second layer applied to an interfacing side of the wearable film dressing, the second layer comprising:
a flexible polymer substrate, and
an electrode array integrated onto the flexible polymer substrate, wherein:
the second layer is configured to be applied to a mammalian epidermis via the first layer, and
an acidic hydrogel interlayer is configured to be disposed between the second layer and the mammalian epidermis.

13. The wearable electroceutical device of claim 12, wherein the electrode array comprises gold electrodes.

14. The wearable electroceutical device of claim 12, wherein the flexible polymer substrate comprises a flexible polyimide substrate.

15. The wearable electroceutical device of claim 12, wherein the electrode array is interdigitated, and wherein the electrode array enables electric potential localization between adjacent fingers of the electrodes in the electrode array.

16. The wearable electroceutical device of claim 12, wherein the acidic hydrogel interlayer comprises a natural and/or synthetic polymer hydrogel with a pH of about 5.

17. The wearable electroceutical device of claim 12, wherein the acidic hydrogel interlayer sensitizes resident bacteria on the mammalian epidermis to electrical excitation.

18. The wearable electroceutical device of claim 17, wherein the resident bacteria comprises at least one of: Staphylococcus epidermidis, Staphylococcus capitis, and Staphylococcus saprophyticus.

19. The wearable electroceutical device of claim 17, wherein a potentiostat connected to the wearable electroceutical device is configured to deliver alternating current to the wearable electroceutical device.

20. The wearable electroceutical device of claim 19, wherein the potentiostat is configured to deliver a voltage of about 1.5V_{ac} to the wearable electroceutical device to elicit a hyperpolarization response in the resident bacteria.
